(19) United States
(12) Patent Application Publication
(10) Pub. No.: US 2011/0224159 A1
(43) Pub. Date: Sep. 15, 2011

(54) BIDESMOSIDIC BETULIN AND BETULINIC ACID DERIVATIVES AND USES THEREOF AS ANTITUMOR AGENTS

(75) Inventors: André Pichette, Chicoutimi (CA); Jean Legault, Chicoutimi (CA); Charles Gauthier, Peribonka (CA)

(73) Assignee: Universite du Quebec a Chicoutime, Chicoutimi (CA)

(21) Appl. No.: 13/063,058

(22) PCT Filed: Sep. 10, 2009
(86) PCT No.: PCT/CA2009/001255

§ 371 Date: May 12, 2011

Related U.S. Application Data
(60) Provisional application No. 61/095,815, filed on Sep. 10, 2008.

(51) Int. Cl. A61K 31/704; C07H 15/24; G01N 33/574; A61P 35/00

(52) U.S. Cl. 514/33; 536/18.1; 435/7.23

(57) ABSTRACT

The instant application is directed to bidesmosidic betulin and betulinic acid saponin derivatives of formula (I), and use thereof as antitumor agents. In particular, said compounds are effective in treating lung carcinomas, colorectal adenocarcinomas, breast adenocarcinomas, and prostate adenocarcinomas. Methods of synthesizing said compounds through selective glycosylation of the C-28 and C-3 position, and diagnostic methods for identifying tumours suitable for treatment by said compounds are also disclosed.

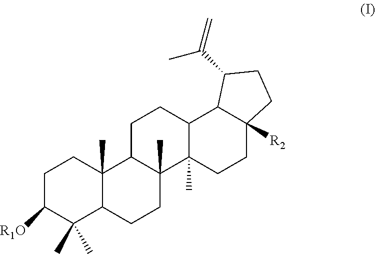

### CROSS REFERENCE TO RELATED APPLICATIONS

This application is a National Entry Application of PCT application no PCT/CA2009/* filed on Sep. 10, 2009 and published in English under PCT Article 21(2), which itself claims priority on U.S. provisional application Ser. No. 61,095,815, filed on Sep. 10, 2008. All documents above are incorporated herein in their entirety by reference.

### STATEMENT REGARDING FEDERALLY SPONSORED RESEARCH OR DEVELOPMENT

N/A.

### FIELD OF THE INVENTION

The invention relates to cancer prevention and/or treatment, and more particularly to bidesmosidic betulin and betulinic acid saponin derivatives and uses thereof as antitumor agents.

### BACKGROUND OF THE INVENTION

One-third of all individuals in the United States will develop cancer during their life. Although the five-year survival rate has risen dramatically as a result of progress in early diagnosis and therapy, cancer still remains second only to cardiac disease as a cause of death in the United States. Twenty percent of Americans die from cancer, half due to lung, breast, and colon-rectal cancer, and skin cancer remains a serious health hazard. Currently available therapies such as chemotherapy and radiotherapy are not effective against all types of cancer and have undesirable side effects (high toxicity). Therefore, there is a great need to develop effective antitumor agents having reduced side effects.

The present description refers to a number of documents, the content of which is herein incorporated by reference in their entirety.

### SUMMARY OF THE INVENTION

In the studies presented herein, the inventors described the synthesis of bidesmosidic betulin and betulinic acid saponin derivatives, and showed that these compounds inhibit the proliferation of various types of tumor cells.

Accordingly, in accordance with one aspect of the present invention, there is provided a compound of formula (I):

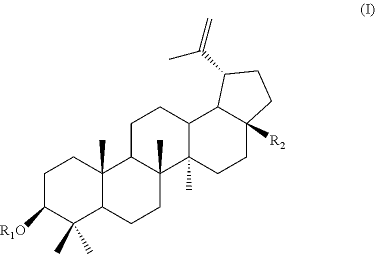

wherein when R₁ is α-L-arabinopyranose, R₂ is CH₂O-β-D-glucopyranose; and when R₁ is α-L-rhamnopyranose, R₂ is CH₂O-β-D-glucopyranose, COO-β-D-glucopyranose, CH₂O-β-rhamnopyranose or COO-β-L-rhamnopyranose, or a pharmaceutically acceptable salt thereof.

In an embodiment, R₁ is α-L-arabinopyranose and R₂ is CH₂O-β-D-glucopyranose.

In another embodiment, R₁ is α-L-rhamnopyranose and R₂ is CH₂O-β-D-Glucopyranose.

In another embodiment, R₁ is α-L-rhamnopyranose and R₂ is COO-β-D-glucopyranose.

In another embodiment, R₁ is α-L-rhamnopyranose and R₂ is CH₂O-β-L-rhamnopyranose.

In another embodiment, R₁ is α-L-rhamnopyranose and R₂ is COO-β-L-rhamnopyranose.

In another aspect, the present invention provides a pharmaceutical composition comprising the above-mentioned compound and a pharmaceutically acceptable diluent, carrier or excipient.

In another aspect, the present invention provides a method for treating carcinoma comprising administering to a subject in need thereof an effective amount of a compound of formula (I):

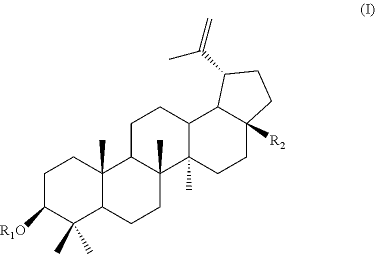

wherein when R₁ is α-L-arabinopyranose, R₂ is CH₂O-β-D-glucopyranose or COO-β-D-glucopyranose; when R₁ is α-L-rhamnopyranose, R₂ is CH₂O-β-D-glucopyranose, COO-β-D-glucopyranose, CH₂O-β-L-rhamnopyranose or COO-β-L-rhamnopyranose; and when R₁ is β-D-glucopyranose, R₂ is CH₂O-β-D-glucopyranose or COO-β-D-glucopyranose, or a pharmaceutically acceptable salt thereof.

In an embodiment, the above-mentioned administration is parenteral or systemic.

In another embodiment, the above-mentioned administration is at a tumour site.

In an embodiment, the above-mentioned administration is in a dosage of about 0.5 mg/kg to about 50 mg/kg. In a further embodiment, the above-mentioned administration is in a dosage of about 4 mg/kg to about 40 mg/kg.

In another aspect, the present invention provides a use of a compound of formula (I):

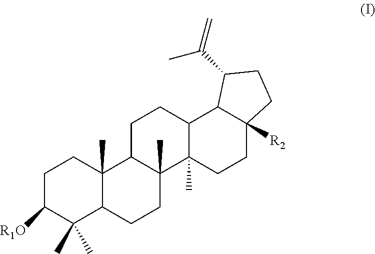

wherein when R₁ is α-L-arabinopyranose, R₂ is CH₂O-β-D-glucopyranose or COO-β-D-glucopyranose; when R₁ is α-L-rhamnopyranose, R₂ is CH₂O-β-D-glucopyranose, COO-β-D-glucopyranose, CH₂O-β-L-rhamnopyranose or COO-β-L-rhamnopyranose; and when R₁ is β-D-glucopyranose, R₂ is CH₂O-β-D-glucopyranose or COO-β-D-glucopyranose, or a pharmaceutically acceptable salt thereof, for treating a carcinoma in a subject.

In another aspect, the present invention provides a use of a compound of formula (I):

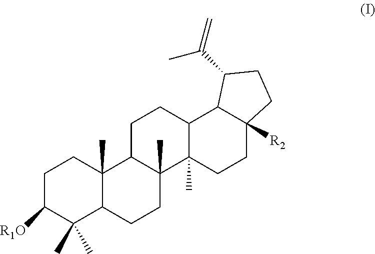

wherein when R₁ is α-L-arabinopyranose, R₂ is CH₂O-β-D-glucopyranose or COO-β-D-glucopyranose; when R₁ is α-L-rhamnopyranose, R₂ is CH₂O-β-D-glucopyranose, COO-β-D-glucopyranose, CH₂O-β-L-rhamnopyranose or COO-β-L-rhamnopyranose; and when R₁ is β-D-glucopyranose, R₂ is CH₂O-β-D-glucopyranose or COO-β-D-glucopyranose, or a pharmaceutically acceptable salt thereof, for the preparation of a medicament for treating a carcinoma in a subject.

In another aspect, the present invention provides a compound of formula (I):

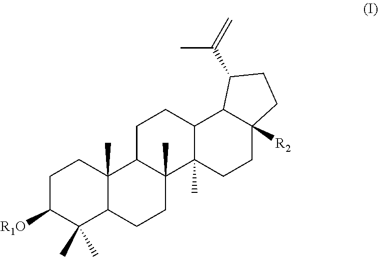

wherein when R₁ is α-L-arabinopyranose, R₂ is CH₂O-β-D-glucopyranose or COO-β-D-glucopyranose; when R₁ is α-L-rhamnopyranose, R₂ is CH₂O-β-D-glucopyranose, COO-β-D-glucopyranose, CH₂O-β-L-rhamnopyranose or COO-β-L-rhamnopyranose; and when R₁ is β-D-glucopyranose, R₂ is CH₂O-β-D-glucopyranose or COO-β-D-glucopyranose, or a pharmaceutically acceptable salt thereof, for treating a carcinoma in a subject.

In an embodiment, the above-mentioned carcinoma is lung carcinoma, colorectal adenocarcinoma, breast adenocarcinoma, or prostate adenocarcinoma.

In an embodiment, the above-mentioned carcinoma is breast adenocarcinoma and wherein when R₁ is α-L-arabinopyranose, R₂ is CH₂O-β-D-glucopyranose or COO-β-D-glucopyranose; when R₁ is α-L-rhamnopyranose, R₂ is CH₂O-β-D-glucopyranose, COO-β-D-glucopyranose, CH₂O-β-L-rhamnopyranose or COO-β-L-rhamnopyranose; and when R₁ is β-D-glucopyranose, R₂ is CH₂O-β-D-glucopyranose.

In another embodiment, the above-mentioned carcinoma is lung carcinoma, and wherein when R₁ is α-L-arabinopyranose, R₂ is COO-β-D-glucopyranose; and when R₁ is α-L-rhamnopyranose, R₂ is CH₂O-β-D-glucopyranose, COO-β-D-glucopyranose, CH₂O-β-L-rhamnopyranose or COO-β-L-rhamnopyranose.

In another embodiment, the above-mentioned carcinoma is prostate adenocarcinoma, and wherein when R₁ is α-L-arabinopyranose, R₂ is CH₂O-β-D-glucopyranose or COO-β-D-glucopyranose; when R₁ is α-L-rhamnopyranose, R₂ is CH₂O-β-D-glucopyranose, COO-β-D-glucopyranose, CH₂O-β-L-rhamnopyranose or COO-β-L-rhamnopyranose; and when R₁ is β-D-glucopyranose, R₂ is COO-β-D-glucopyranose.

In an embodiment, R₁ is α-L-rhamnopyranose and R₂ is CH₂O-β-L-rhamnopyranose.

In an embodiment, the above-mentioned compound is adapted for parenteral or systemic administration.

In another embodiment, the above-mentioned compound is adapted for administration at a tumor site.

In another aspect, the present invention provides a method of identifying a tumor amenable to treatment with the above-mentioned compound, comprising (i) contacting a sample of cells derived from said tumor with the compound, and (ii) determining the IC₅₀ value of the compound against the cells, wherein an IC₅₀ value of about 50 μM or less is indicative that the tumor is amenable to treatment with said compound.

In another embodiment, the above-mentioned IC₅₀ value is 20 μM or less. In a further embodiment, the above-mentioned IC₅₀ value is 10 μM or less.

In an embodiment, the above-mentioned sample of cells is derived from a biopsy sample from a subject.

In another embodiment, the above-mentioned sample of cells is derived from a biological fluid obtained from a subject.

In another aspect, the present invention provides a method of inhibiting the growth of a carcinoma cell comprising contacting said cell with an effective amount of a compound of formula (I):

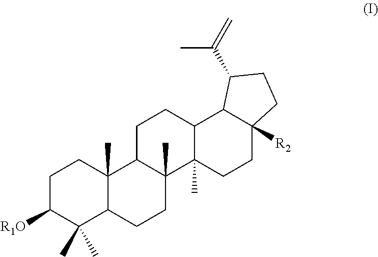

wherein when R₁ is α-L-arabinopyranose, R₂ is CH₂O-β-D-glucopyranose or COO-β-D-glucopyranose; when R₁ is α-L-rhamnopyranose, R₂ is CH₂O-β-D-glucopyranose, COO-β-D-glucopyranose, CH₂O-β-L-rhamnopyranose or COO-β-L-rhamnopyranose; when R₁ is β-D-glucopyranose, R₂ is CH₂O-β-D-glucopyranose or COO-β-D-glucopyranose, or a pharmaceutically acceptable salt thereof.

In an embodiment, the above-mentioned carcinoma cell is a lung carcinoma cell, a colorectal adenocarcinoma cell, a breast adenocarcinoma cell, or a prostate adenocarcinoma cell.

In an embodiment, the above-mentioned carcinoma cell is a breast adenocarcinoma cell and wherein when R₁ is α-L-arabinopyranose, R₂ is CH₂O-β-D-glucopyranose or COO-β-D-glucopyranose; when R₁ is α-L-rhamnopyranose, R₂ is CH₂O-β-D-glucopyranose, COO-β-D-glucopyranose, CH₂O-β-L-rhamnopyranose or COO-β-L-rhamnopyranose; when R₁ is β-D-glucopyranose, R₂ is CH₂O-β-D-glucopyranose.

In another embodiment, the above-mentioned carcinoma cell is a lung carcinoma cell, and wherein when R₁ is α-L-arabinopyranose, R₂ is COO-β-D-glucopyranose; when R₁ is α-L-rhamnopyranose, R₂ is CH₂O-β-D-glucopyranose, COO-β-D-glucopyranose, CH₂O-β-L-rhamnopyranose or COO-β-L-rhamnopyranose; and when R₁ is β-D-glucopyranose, R₂ is CH₂O-β-D-glucopyranose or COO-β-D-glucopyranose.

In another embodiment, the above-mentioned carcinoma cell is a lung carcinoma cell or a prostate adenocarcinoma cell, and wherein when R₁ is α-L-arabinopyranose, R₂ is CH₂O-β-D-glucopyranose or COO-β-D-glucopyranose; when R₁ is α-L-rhamnopyranose, R₂ is CH₂O-β-D-glucopyranose, COO-β-D-glucopyranose, CH₂O-β-L-rhamnopyranose or COO-β-L-rhamnopyranose; when R₁ is β-D-glucopyranose, R₂ is COO-β-D-glucopyranose.

In an embodiment, R₁ is α-L-rhamnopyranose and R₂ is CH₂O-β-L-rhamnopyranose.

In an embodiment, the above-mentioned compound is present in a pharmaceutical composition.

In another aspect, the present invention provides a method for preparing a compound of formula (I):

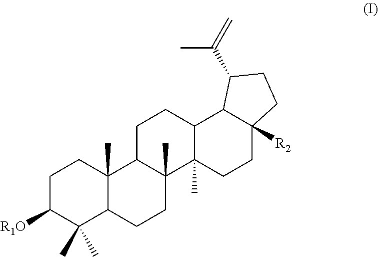

wherein R₁ is α-L-arabinopyranose or α-L-rhamnopyranose, and R₂ is CH₂O-β-D-glucopyranose; said method comprising (a) glycosylating the C-28 position of betulin 3-acetate with a perbenzoylated or peracetylated trichloroacetimidate or trifluorophenylacetimidate glucose donor under the promotion of a Lewis acid to yield a first glycosylated compound; (b) submitting the first glycosylated compound to regioselective deacetylation conditions to cleave the acetyl group at the C-3 position to yield a deacetylated compound; (c) glycosylating the C-3 position of the deacetylated compound with a perbenzoylated or peracetylated trichloroacetimidate or trifluorophenylacetimidate arabinose or rhamnose donor under the promotion of a Lewis acid to yield a second glycosylated compound; and (d) submitting the second glycosylated compound to deacetylation conditions.

In an embodiment, the above-mentioned Lewis acid of (a) is (i) trimethylsilyl trifluoromethanesulfonate (TMSOTf), (ii) tert-butyldimethylsilyl trifluoromethanesulfonate (TBSOTf), (iii) boron trifluoride diethyletherate (BF3-OEt₂), or (iv) any combination of (i) to (iii).

In another embodiment, the above-mentioned Lewis acid of (c) is (i) trimethylsilyl trifluoromethanesulfonate (TMSOTf), (ii) Pert-butyldimethylsilyl trifluoromethanesulfonate (TBSOTf), (iii) boron trifluoride diethyletherate (BF₃-OEt₂), or (iv) any combination of (i) to (iii).

In an embodiment, the above-mentioned regioselective deacetylation conditions comprise (a) acetyl chloride (AcCl) in a solution of CH₂Cl₂/MeOH, (b) para-toluenesulfonic acid monohydrate (TsOH.H₂O) in a solution of CH₂Cl₂/MeOH at 40° C., or (c) Hydrazine hydrate (NH₂NH₂.xH-₂O) in tetrahydrofuran (THF).

In an embodiment, the above-mentioned deacetylation conditions of (d) comprise (i) NaOMe and MeOH (Zemplén deacetylation conditions) or (ii) NaOH in MeOH/tetrahydrofuran/H₂O.

In an embodiment, the above-mentioned perbenzoylated or peracetylated trichloroacetimidate or trifluorophenylacetimidate glucose donor is 2,3,4,6-tetra-O-benzoyl-α-D-glucopyranosyl trichloroacetimidate.

In an embodiment, the above-mentioned perbenzoylated or peracetylated trichloroacetimidate or trifluorophenylacetimidate arabinose donor is 2,3,4-tri-O-benzoyl-β-L-arabinopyranosyl trichloroacetimidate.

In an embodiment, the above-mentioned perbenzoylated or peracetylated trichloroacetimidate or trifluorophenylacetimidate rhamnose donor is 2,3,4-tri-O-benzoyl-α-L-rhamnopyranosyl trichloroacetimidate.

In another aspect, the present invention provides a method for preparing a compound of formula (I):

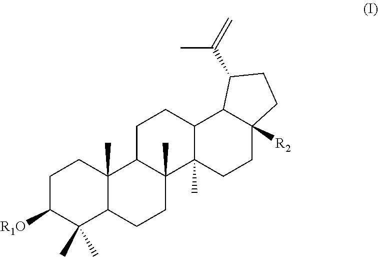

wherein R₁ is α-L-arabinopyranose or α-L-rhamnopyranose, and R₂ is COO-β-D-glucopyranose; said method comprising (a) glycosylating the C-28 position of betulinic acid with a perbenzoylated or peracetylated bromide glucose donor under phase-transfer conditions to yield a first glycosylated compound; (b) glycosylating the C-3 position of the first glycosylated compound with a perbenzoylated or peracetylated trichloroacetimidate or trifluorophenylacetimidate rhamnose or arabinose donor under the promotion of a Lewis acid to yield a second glycosylated compound; and (c) submitting the second glycosylated compound to deacetylation conditions.

In an embodiment, the above-mentioned deacetylation conditions comprises (i) NaOMe and MeOH (Zemplén deacetylation conditions) or (ii) NaOH in MeOH/tetrahydrofuran/H₂O.

In an embodiment, the above-mentioned NaOH is at about 0.5 N.

In an embodiment, the above-mentioned phase-transfer conditions comprises K₂CO₃, a quaternary ammonium salt, CH₂Cl₂/H₂O and reflux.

In a further embodiment, the above-mentioned quaternary ammonium salt is Bu₄NI, Bu₄NBr, Bu₄NCl, Aliquat™ 100, Aliquat™ 175, Aliquat™ 336 or Aliquat™ HTA-1.

In an embodiment, the above-mentioned perbenzoylated or peracetylated bromide glucose donor is 2,3,4,6-tetra-O-benzoyl-α-D-glucopyranosyl bromide.

In an embodiment, the above-mentioned perbenzoylated or peracetylated trichloroacetimidate or trifluorophenylacetimidate arabinose donor is 2,3,4-tri-O-benzoyl-β-L-arabinopyranosyl trichloroacetimidate.

In an embodiment, the above-mentioned perbenzoylated or peracetylated trichloroacetimidate or trifluorophenylacetimidate rhamnose donor is 2,3,4-tri-O-benzoyl-α-L-rhamnopyranosyl trichloroacetimidate.

In another aspect, the present invention provides a method for preparing a compound of formula (I):

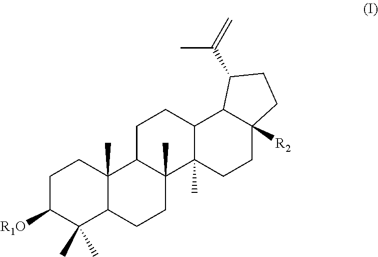

wherein when R₁ is β-D-glucopyranose, R₂ is COO-β-D-glucopyranose or CH₂O-β-D-glucopyranose; when R₁ is α-L-rhamnopyranose, R₂ is COO-β-L-rhamnopyranose or CH₂O-β-L-rhamnopyranose; said method comprising (a) glycosylating the C-3 and C-28 positions of betulin or betulinic acid with a perbenzoylated or peracetylated trichloroacetimidate or trifluorophenylacetimidate glucose or rhamnose donor under the promotion of a Lewis acid via a Schmidt's inverse procedure to yield a glycosylated compound; and (b) submitting the glycosylated compound to deacetylation conditions.

In an embodiment, the above-mentioned Lewis acid is (i) trimethylsilyl trifluoromethanesulfonate (TMSOTf), (ii) tert-butyldimethylsilyl trifluoromethanesulfonate (TBSOTf), (iii) boron trifluoride diethyletherate (BF₃-OEt₂), or (iv) any combination of (i) to (iii).

In an embodiment, the above-mentioned deacetylation conditions comprises (i) NaOMe and MeOH (Zemplén deacetylation conditions) or (ii) NaOH in MeOH/tetrahydrofuran/H₂O.

In an embodiment, the above-mentioned step (a) glycosylates the C-3 and C-28 positions of betulin.

In another embodiment, the above-mentioned step (a) glycosylates the C-3 and C-28 positions of betulinic acid.

In an embodiment, the above-mentioned perbenzoylated or peracetylated trichloroacetimidate or trifluorophenylacetimidate glucose donor is 2,3,4,6-tetra-O-benzoyl-α-D-glucopyranosyl trichloroacetimidate.

In an embodiment, the above-mentioned perbenzoylated or peracetylated trichloroacetimidate or trifluorophenylacetimidate rhamnose donor is 2,3,4-tri-O-α-L-rhamnopyranosyl trichloroacetimidate.

In an embodiment, the above-mentioned Schmidt's inverse procedure comprises pre-mixing said betulin or betulinic acid with said Lewis acid before adding said perbenzoylated or peracetylated trichloroacetimidate or trifluorophenylacetimidate glucose or rhamnose donor.

In a further embodiment, the above-mentioned addition of the perbenzoylated or peracetylated trichloroacetimidate or trifluorophenylacetimidate glucose or rhamnose donor is performed at a temperature of between about −78° C. to about 25° C. In a further embodiment, the above-mentioned addition of the perbenzoylated or peracetylated trichloroacetimidate or trifluorophenylacetimidate glucose or rhamnose donor is performed at a temperature of about −10° C.

Other objects, advantages and features of the present invention will become more apparent upon reading of the following non-restrictive description of specific embodiments thereof, given by way of example only with reference to the accompanying drawings.

### BRIEF DESCRIPTION OF THE DRAWINGS

In the appended drawings:

FIG. 1 presents the chemical structure of betulin (1), betulinic acid (2) and natural bidesmosidic betulinic acid saponin (3);

FIG. 2 presents the glycosyl donors (4-8) used for the synthesis of bidesmosides;

FIG. 3 presents an attempt to synthesize bidesmosidic betulin saponins (12a, 12b). A donor 4 (1.5 equiv), TMSOTf, CH₂Cl₂, 4 Å MS, room temperature (rt), 16 h; B: inverse procedure, donor 4 (1.5 equiv), TMSOTf, CH₂Cl₂, 4 Å MS, −10° C. to rt, 2.5 h; C: donor 6 (1.5 equiv), AgOTf, CH₂Cl₂, 4 Å MS, −78 to 0° C., 2 h; D: donor 5 (1.5 equiv), BF₃.OEt₂, CH₂Cl₂, 4 Å MS, −78 to 0° C., 24 h; E: donor 6 (1.3 equiv), K₂CO₃, Bu₄NBr, CH₂Cl₂/H₂O 1:1, reflux, 5 h.;

FIG. 4 presents the method used for the synthesis of bidesmosidic betulin saponins (16a, 16b);

FIG. 5 presents the method used for the synthesis of bidesmosidic betulinic acid saponins (3, 19);

FIG. 6 presents an attempt to synthesize benzoylated bidesmosidic betulinic saponins (20). A: donor 4 (1.5 equiv), TMSOTf, CH₂Cl₂, 4 Å MS, room temperature (rt), 16 h; B: inverse procedure, donor 4 (3 equiv), TMSOTf, CH₂Cl₂, 4 Å MS, −10° C. to rt, 3.5 h; C: donor 6 (1.5 equiv), Ag₂O, CH₃CN/CH₂Cl₂, 4 Å MS, rt, 4 d; D: donor 6 (1.5 equiv), AgOTf, CH₂Cl₂, 4 Å MS, 0 to 16° C., 2 h.; and

FIG. 7 presents the method used for the synthesis of bidesmosidic saponins (21a, 21b, 22a, 22b) by the Schmidt's “inverse procedure”.

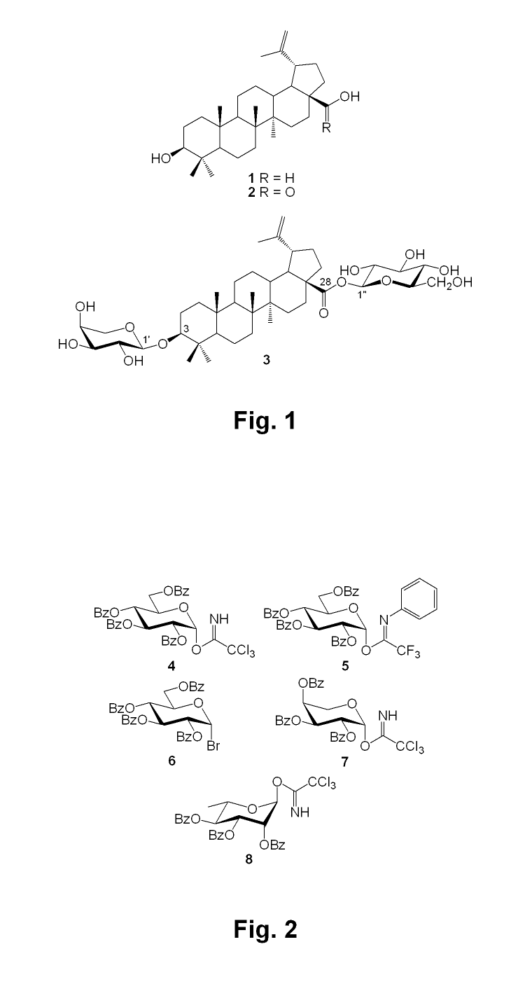

### DESCRIPTION OF ILLUSTRATIVE EMBODIMENTS

The present inventors have shown that bidesmosidic betulin and betulinic acid saponin derivatives, which may be represented by formula (I) below, reduce or inhibit the growth of various types of tumor cells, and thus may be useful for the prevention and treatment of cancers such as carcinomas.

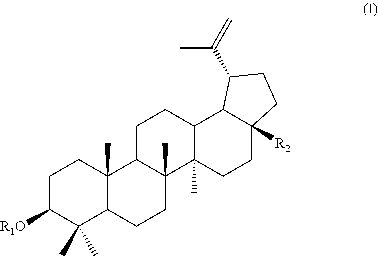

wherein R₁ is a monosaccharide-based residue and R₂ is an ester or ether of a monosaccharide-based residue, with the proviso that when R₁ is β-D-glucopyranose, R₂ is not CH₂O-β-D-glucopyranose or COO-β-D-glucopyranose and when R₁ is α-L-arabinopyranose, R₂ is not COO-β-D-glucopyranose.

In another embodiment, R₁ is a glucose-, rhamnose- or arabinose-based residue and R₂ is an ester or ether of a glucose- or rhamnose-based residue.

In another embodiment, R₁ is a monosaccharide-based residue and R₂ is an ester or ether of a monosaccharide-based residue, and either R₁ is a rhamnose-based residue or R₂ is an ester or ether of a rhamnose-based residue.

Accordingly, in a first aspect, the present invention provides a compound of formula (I), wherein when R₁ is α-L-arabinopyranose, R₂ is CH₂O-β-D-glucopyranose; when R₁ is α-L-rhamnopyranose, R₂ is CH₂O-β-D-glucopyranose, COO-β-D-glucopyranose, CH₂O-β-L-rhamnopyranose or COO-β-L-rhamnopyranose, or a pharmaceutically acceptable salt thereof.

The terms “pharmaceutically acceptable salts” refer to salts of compounds of the present invention that are pharmacologically acceptable and substantially non-toxic to the subject to which they are administered. More specifically, these salts retain the biological effectiveness and properties of the compounds of the invention and are formed from suitable non-toxic organic or inorganic acids or bases.

### Esters

The present invention relates to the compounds of the invention as hereinbefore defined as well as to the esters thereof. The term “ester(s)”, as employed herein, refers to compounds of the invention or salts thereof in which hydroxy groups have been converted to the corresponding esters using, for example, inorganic or organic anhydrides, acids, or acid chlorides. Esters for use in pharmaceutical compositions will be pharmaceutically acceptable esters, but other esters may be useful in the production of the compounds of the invention. For instance esters can be prepared on alcool groups of the sugar moieties.

The term “pharmaceutically acceptable esters” refers to esters of the compounds of the present invention that are pharmacologically acceptable and substantially non-toxic to the subject to which they are administered. More specifically, these esters retain the biological effectiveness and properties of the compounds of the invention and act as prodrugs which, when absorbed into the bloodstream of a warm-blooded animal, cleave in such a manner as to produce the parent alcohol compound.

Esters of the present compounds include among others the following groups (1) carboxylic acid esters obtained by esterification of the hydroxy groups, in which the non-carbonyl moiety of the carboxylic acid portion of the ester grouping is selected from straight or branched chain alkyl (for example, acetyl, n-propyl, t-butyl, n-butyl, methyl, ethyl, propyl, isopropyl, butyl, isobutyl or pentyl), alkoxyalkyl (for example, methoxymethyl, acetoxymethyl and 2,2-dimethylpropionyloxymethyl), aralkyl (for example, benzyl), aryloxyalkyl (for example, phenoxymethyl), aryl (for example, phenyl optionally substituted with, for example, halogen, C₁₋₄ alkyl, or C₁₋₄ alkoxy or amino); (2) sulfonate esters, such as alkyl- or aralkylsulfonyl (for example, methanesulfonyl); (3) amino acid esters (for example, L-valyl or L-isoleucyl); (4) phosphonate esters; (5) mono-, di- or triphosphate esters (including phosphoramidic cyclic esters). The phosphate esters may be further esterified by, for example, a C₁₋₂₀ alcohol or reactive derivative thereof, or by a 2,3-di(C₆₋₂₄)acyl glycerol.

Further information concerning examples of and the use of esters for the delivery of pharmaceutical compounds is available in Design of Prodrugs. Bundgaard H ed. (Elsevier, 1985). See also, H. Ansel et. al., Pharmaceutical Dosage Forms and Drug Delivery Systems (6th Ed. 1995) at pp. 108-109; Krogsgaard-Larsen, et. al., Textbook of Drug Design and Development (2d Ed. 1996) at pp. 152-191; and Wermuth, C. G. Practice of medicinal chemistry. 2^{nd} Edition, Elsevier Academic Press, San Diego, USA, 2003, at pages 617-630.

The compounds of this invention may be esterified by a variety of conventional procedures including reacting the appropriate anhydride, carboxylic acid or acid chloride with the alcohol group of a compound of this invention. For example, an appropriate anhydride may be reacted with an alcohol in the presence of a base, such as 1,8-bis[dimethylamino]naphthalene or N,N-dimethylaminopyridine, to facilitate acylation. Also, an appropriate carboxylic acid can be reacted with the alcohol in the presence of a dehydrating agent such as dicyclohexylcarbodiimide, 1-[3-dimethylaminopropyl]-3-ethylcarbodiimide or other water soluble dehydrating agents which are used to drive the reaction by the removal of water, and, optionally, an acylation catalyst. Esterification can also be effected using the appropriate carboxylic acid. Reaction of an acid chloride with the alcohol can also be carried out. When a compound of the invention contains a number of free hydroxy group, those groups not being converted into a prodrug functionality may be protected (for example, using a tert-butyldimethylsilyl group), and later deprotected. Also, enzymatic methods may be used to selectively phosphorylate or dephosphorylate alcohol functionalities. One skilled in the art would readily know how to successfully carry out these as well as other known methods of esterification of alcohols.

Esters of the compounds of the invention may form salts. Where this is the case, this is achieved by conventional techniques as described above.

### Solvates

One or more compounds of the invention may exist in unsolvated as well as solvated forms with solvents such as water, ethanol, and the like, and it is intended that the invention embrace both solvated and unsolvated forms.

“Solvate” means a physical association of a compound of this invention with one or more solvent molecules. This physical association involves varying degrees of ionic and covalent bonding, including hydrogen bonding. In certain instances the solvate will be capable of isolation, for example when one or more solvent molecules are incorporated in the crystal lattice of the crystalline solid. “Solvate” encompasses both solution-phase and isolatable solvates. Solvates for use in pharmaceutical compositions will be pharmaceutically acceptable solvates, but other solvates may be useful in the production of the compounds of the invention.

As used herein, the term “pharmaceutically acceptable solvates” means solvates of compounds of the present invention that are pharmacologically acceptable and substantially non-toxic to the subject to which they are administered. More specifically, these solvates retain the biological effectiveness and properties of the compounds of the invention and are formed from suitable non-toxic solvents.

Non-limiting examples of suitable solvates include ethanolates, methanolates, and the like, as well as hydrates, which are solvates wherein the solvent molecules are H₂O.

Preparation of Solvates is Generally Known. Thus, for Example, M. Caira et al., J. Pharmaceutical Sci., 93(3), 601-611 (2004) describe the preparation of the solvates of the antifungal fluconazole in ethyl acetate as well as from water. Similar preparations of solvates, hemisolvate, hydrates and the like are described by E. C. van Tonder et al, AAPS Pharm Sci Tech., 5(1), article 12 (2004); and A. L. Bingham et al, Chem. Commun., 603-604 (2001); Wermuth, C. G. Practice of medicinal chemistry. 2^{nd} Edition, Elsevier Academic Press, San Diego, USA, 2003,768 pp.

A typical non-limiting process for preparing a solvate involves dissolving the inventive compound in desired amounts of the desired solvent (organic or water or mixtures thereof) at a higher than ambient temperature, and cooling the solution at a rate sufficient to form crystals which are then isolated by standard methods. Analytical techniques such as, for example IR spectroscopy, can be used to show the presence of the solvent (or water) in the crystals as a solvate (or hydrate).

### Isomers, Tautomers and Polymorphs:

As used herein, the term “isomers” refers to optical isomers (enantiomers), diastereoisomers as well as the other known types of isomers.

Some of the compounds of the invention may have at least one asymmetric carbon atoms and can therefore exist in the form of optically pure enantiomers (optical isomers), as racemates and as mixtures thereof. Some of the compounds may have at least two asymmetric carbon atoms and can therefore exist in the form of pure diastereoisomers and as mixtures thereof.

It is to be understood, that, unless otherwise specified, the present invention embraces the racemates, the enantiomers and/or the diastereoisomers of the compounds of the invention as well as mixtures thereof.

The synthesis of optically active forms of the compounds of the invention may be carried out by standard techniques of organic chemistry well known in the art, for example by resolution of the racemic form by recrystallisation techniques, by chiral synthesis, by enzymatic resolution, by biotransformation or by chromatographic separation. More specifically, diastereomeric mixtures can be separated into their individual diastereoisomers on the basis of their physical chemical differences by methods well known to those skilled in the art, such as, for example, by chromatography and/or fractional crystallization. Enantiomers can be separated, for example, by converting the enantiomeric mixture into a diastereomeric mixture by reaction with an appropriate optically active compound (e.g., chiral auxiliary such as a chiral alcohol), separating the diastereoisomers and converting (e.g., hydrolyzing) the individual diastereoisomers to the corresponding pure enantiomers.

In addition, the present invention embraces all geometric and positional isomers. For example, if a compound of the invention incorporates a double bond or a fused ring, both the cis- and trans-forms, as well as mixtures, are embraced within the scope of the invention.

Within the present invention it is to be understood that a compound of the invention may exhibit the phenomenon of tautomerism and that the formula drawings within this specification can represent only one of the possible tautomeric forms. It is to be understood that the invention encompasses any tautomeric form and is not to be limited merely to any one tautomeric form utilized within the formula drawings.

It is also to be understood that certain compounds of the invention may exhibit polymorphism, and that the present invention encompasses all such forms.

As used herein the term “compound of formula I” is meant to include D-enantiomers, L-enantiomers and racemates of the compound of formula I.

In another aspect, the present invention provides a method for treating carcinoma comprising administering to a subject in need thereof an effective amount of a compound of formula (I) illustrated above, wherein when R₁ is α-L-arabinopyranose, R₂ is CH₂O-β-D-glucopyranose or COO-β-D-glucopyranose; when R₁ is α-L-rhamnopyranose, R₂ is CH₂O-β-D-glucopyranose, COO-β-D-glucopyranose, CH₂O-β-L-rhamnopyranose or COO-β-L-rhamnopyranose; when R₁ is β-D-glucopyranose, R₂ is CH₂O-β-D-glucopyranose or COO-β-D-glucopyranose, or a pharmaceutically acceptable salt thereof.

In another aspect, the present invention provides a use of a compound of formula (I) illustrated above, wherein when R₁ is α-L-arabinopyranose, R₂ is CH₂O-β-D-glucopyranose or COO-β-D-glucopyranose; when R₁ is α-L-rhamnopyranose, R₂ is CH₂O-β-D-glucopyranose, COO-β-D-glucopyranose, CH₂O-β-L-rhamnopyranose or COO-β-L-rhamnopyranose; when R₁ is β-D-glucopyranose, R₂ is CH₂O-β-D-glucopyranose or COO-β-D-glucopyranose, or a pharmaceutically acceptable salt thereof, for treating carcinoma in a subject.

In another aspect, the present invention provides a use of a compound of formula (I) illustrated above, wherein when R₁ is α-L-arabinopyranose, R₂ is CH₂O-β-D-glucopyranose or COO-β-D-glucopyranose; when R₁ is α-L-rhamnopyranose, R₂ is CH₂O-β-D-glucopyranose, COO-β-D-glucopyranose, CH₂O-β-L-rhamnopyranose or COO-β-L-rhamnopyranose; when R₁ is β-D-glucopyranose, R₂ is CH₂O-β-D-glucopyranose or COO-β-D-glucopyranose, or a pharmaceutically acceptable salt thereof, for the preparation of a medicament for treating carcinoma in a subject.

The term “subject” or “patient” as used herein refers to an animal, preferably a mammal, and most preferably a human who is the object of treatment, observation or experiment.

An “effective amount” (e.g., a “therapeutically and/or prophylactically effective amount”) refers to an amount effective, at dosages and for periods of time necessary, to achieve the desired prophylactic and/or therapeutic result, such as a prevention or reduction of tumor growth and in turn a reduction in cancer-related disease or progression. An effective amount of the above-mentioned compound may vary according to factors such as the disease state, age, sex, and weight of the individual, and the ability of the compound to elicit a desired response in the individual. Dosage regimens may be adjusted to provide the optimum prophylactic/therapeutic response. An effective amount is also one in which any toxic or detrimental effects of the compound are outweighed by the beneficial effects.

The term “treating cancer” or “treatment of cancer” as used herein includes at least one of the following features: alleviation of a symptom associated with the cancer, a reduction in the extent of the cancer (e.g., a reduction in tumor growth or size), a stabilization of the state of the cancer (e.g., an inhibition of tumor growth).

The term “preventing cancer” or “prevention of cancer” as used herein includes at least one of the following features: a prevention of further spread of the cancer (e.g., a metastasis), a prevention of the occurrence or recurrence of a cancer, a delaying or retardation of the progression of the cancer (e.g., a reduction in tumor growth) or an improvement in the state of the cancer (e.g., a reduction in tumor size).

The compounds of the present invention can be orally or parenterally and stably administered to human and animals to act as, for instance, a drug or a quasi-drug. In this respect, examples of parenteral administration include intravenous injection, intra-arterial injection, intramuscular injection, subcutaneous injection, intracutaneous injection, intraperitoneal injection, intra-spinal injection, peridural injection, percutaneous administration, perpulmonary administration, pernasal administration, perintestinal administration, administration through oral cavity and permucosal administration and examples of dosage forms used in such parenteral administration routes include injections, suppositories (e.g., rectal suppositories, urethral suppositories and vaginal suppositories), liquids for external use (e.g., injections, gargles, mouth washes, fomentations, inhalants, sprays, aerosols, enema, paints, cleaning agents, disinfectants, nasal drops and ear drops), cataplasms, percutaneous absorption tapes, external preparations for the skin, ointments (e.g., pastes, liniments and lotions). In addition, examples of pharmaceutical preparations for oral administration include tablets for internal use (e.g., uncoated tablets, sugar-coated tablets, coating tablets, enteric coated tablets and chewable tablets), tablets administered to oral cavity (e.g., buccal preparations, sublingual tablets, troches and adhesive tablets), powders, capsules (e.g., hard capsules and soft capsules), granules (e.g., coated granules, pills, troches, liquids preparations or pharmaceutically acceptable sustained release pharmaceutical preparations). Specific examples of liquid preparations capable of being orally administered are solutions for internal use, shake mixtures, suspensions, emulsions, syrups, dry syrups, elixirs, infusion and decoction and lemonades.

The above-mentioned compound of formula (I) may be administered in the form of a prodrug. The term “prodrug” as used herein is defined as a compound that is administered in an inactive or significantly less active form and which is metabolized in vivo (e.g., after administration to a subject) into an active or more active metabolite. The prodrug may, for example, have a better bioavailability or enhanced solubility in water, may be less toxic and/or may facilitate targeting of the drug to the desired site (e.g., tissue or organ in which tumor cells are present).

The invention also relates to a composition (e.g., a pharmaceutical composition, an antitumor composition) comprising the above-mentioned compound of formula (I) and a pharmaceutically acceptable diluent, carrier or excipient. As used herein “pharmaceutically acceptable carrier” or “diluent” or “excipient” includes any and all solvents, dispersion media, coatings, antibacterial and antifungal agents, isotonic and absorption delaying agents, and the like that are physiologically compatible. In one embodiment, the carrier is suitable for parenteral administration. Alternatively, the carrier can be suitable for intravenous, intraperitoneal, intramuscular, sublingual or oral administration. Pharmaceutically acceptable carriers include sterile aqueous solutions or dispersions and sterile powders for the extemporaneous preparation of sterile injectable solutions or dispersion. The use of such media and agents for pharmaceutically active substances is well known in the art (Rowe et al., Handbook of pharmaceutical excipients, 2003, 4^{th} edition, Pharmaceutical Press, London UK). Except insofar as any conventional media or agent is incompatible with the active compound, use thereof in the pharmaceutical compositions of the invention is contemplated. Supplementary active compounds can also be incorporated into the compositions.

Pharmaceutical composition within the scope of the present invention desirably contain the active agent (the above-mentioned compound of formula (I)) in an amount effective to achieve the desired therapeutic effect while avoiding adverse side effects. Pharmaceutically acceptable preparations and salts of the active agent are within the scope of the present invention and are well known in the art. The amount of the therapeutic or pharmaceutical composition which is effective in the treatment of a particular disease, disorder or condition will depend on the nature and severity of the disease, the target site of action, the patient's weight, special diets being followed by the patient, concurrent medications being used, the administration route and other factors that will be recognized by those skilled in the art. The dosage will be adapted by the clinician in accordance with conventional factors such as the extent of the disease and different parameters from the patient. Typically, 0.001 to 100 mg/kg/day will be administered to the subject. Effective doses may be extrapolated from dose response curves derived from in vitro or animal model test systems. For example, in order to obtain an effective mg/kg dose for humans based on data generated from mice studies, the effective mg/kg dosage in rat is divided by 12.3.

The pharmaceutical compositions of the present invention can be delivered in a controlled release system. For example, polymeric materials can be used (see Smolen and Ball, Controlled Drug Bioavailability, Drug product design and performance, 1984, John Wiley & Sons; Ranade and Hollinger, Drug Delivery Systems, pharmacology and toxicology series, 2003, 2^{nd} edition, CRRC Press), or a pump may be used (Saudek et al., 1989, N. Engl. J. Med. 321: 574).

Compounds of the present invention may also be delivered to the desired site (e.g., tissue or organ in which tumor cells are present) using targeting moieties, including monoclonal antibodies (e.g., antibodies recognizing a tumor marker) as individual carriers to which the compound molecules are coupled. The compounds of the present invention may also be coupled to a class of biodegradable polymers useful in achieving controlled release of the drug, for example, polylactic acid, polyorthoesters, cross-linked amphipathic block copolymers and hydrogels, polyhydroxy butyric acid and polydihydropyrans.

In an embodiment, the above-mentioned compound of formula (I) or the above-mentioned pharmaceutical composition is for preventing and/or treating cancer (e.g., carcinoma) in a subject.

In a further aspect, the present invention provides a method of preventing or inhibiting tumor cell proliferation (e.g., tumor growth) comprising contacting said cell with an effective amount of the above-mentioned compound. The tumors to which the compound of the present invention can be applied include swellings and true tumors including benign and malignant tumors. Specific examples of such tumors are gliomas such as astrocytoma, glioblastoma, medulloblastoma, oligodendroglioma, ependymoma and choroid plexus papilloma; cerebral tumors such as meningioma, pituitary adenoma, neurioma, congenital tumor, metastatic cerebral tumor; squamous cell carcinoma, lymphoma, a variety of adenomas and pharyngeal cancers resulted from these adenomas such as epipharyngeal cancer, mesopharyngeal cancer and hypopharyngeal cancer; laryngeal cancer, thymoma; mesothelioma such as pleural mesothelioma, peritoneal mesothelioma and pericardial mesothelioma; breast cancers such as thoracic duct cancer, lobular carcinoma and papillary cancer; lung cancers such as small cell carcinoma, adenocarcinoma, squamous cell carcinoma, large cell carcinoma and adenosquamous carcinoma; gastric carcinoma; esophageal carcinomas such as cervical esophageal carcinomas, thoracic esophageal carcinomas and abdominal esophageal carcinomas; carcinomas of large intestine such as rectal carcinoma, S-like (sigmoidal) colon carcinoma, ascending colon carcinoma, lateral colon carcinoma, cecum carcinoma and descending colon carcinoma; hepatomas such as hepatocellular carcinoma, intrahepatic hepatic duct carcinoma, hepatocellular blastoma and hepatic duct cystadenocarcinoma; pancreatic carcinoma; pancreatic hormone-dependent tumors such as insulinoma, gastrinoma, VIP-producing adenoma, extrahepatic hepatic duct carcinoma, hepatic capsular carcinoma, pedal carcinoma, renal pelvic and uretal carcinoma; urethral carcinoma; renal cancers such as renal cell carcinoma (Grawitz tumor), Wilms' tumor (nephroblastoma) and renal angiomyolipoma; testicular cancers or germ cell tumors such as seminoma, embryonal carcinoma, vitellicle tumor, choriocarcinoma and teratoma; prostatic cancer, bladder cancer, carcinoma of vulva; hysterocarcinomas such as carcinoma of uterine cervix, uterine corpus cancer and solenoma; hysteromyoma, uterine sarcoma, villous diseases, carcinoma of vagina; ovarian germ cell tumors such as dysgerminoma, vitellicle tumor, premature teratoma, dermoidal cancer and ovarian tumors such as ovarian cancer; melanomas such as nevocyte and melanoma; skin lymphomas such as mycosis fungoides, skin cancers such as endoepidermal cancers resulted from skin cancers, prodrome or the like and spinocellular cancer, soft tissue sarcomas such as fibrous histiocytomatosis, liposarcoma, rhabdomyosarcoma, leiomyosarcoma, synovial sarcoma, sarcoma fibroplasticum (fibrosarcoma), neurioma, hemangiosarcoma, fibrosarcoma, neurofibrosarcoma, perithelioma (hemangiopericytoma) and alveolar soft part sarcoma, lymphomas such as Hodgkin lymphoma and non-Hodgkin lymphoma, myeloma, plasmacytoma, acute myelocytic (myeloid) leukemia and chronic myeloid leukemia, leukemia such as adult T-cell leukemic lymphoma and chronic lymphocytic leukemia, chronic myeloproliferative diseases such as true plethora, essential thrombocythemia and idiopathic myelofibrosis, lymph node enlargement (or swelling), tumor of pleural effusion, ascitic tumor, other various kinds of adenomas, lipoma, fibroma, hemangeoma, myoma, fibromyoma and endothelioma. In an embodiment, the above-mentioned tumor cell is a carcinoma cell. In a further embodiment, the above-mentioned carcinoma cell is a lung carcinoma cell, a colorectal adenocarcinoma cell, a breast adenocarcinoma cell, or a prostate adenocarcinoma cell.

The terms “biological sample” are meant to include any tissue or material derived from a living or dead (human) that may contain tumor cells. Samples include, without being so limited, any tissue or material such as blood or fraction thereof, tissue biopsies (e.g., lung, prostate, kidney, skin, stomach, intestine, liver, lymph nodes, pancreas, breast, etc.), bronchial aspiration, sputum, saliva or urine from test patients (suspected cancer patients and control patients) or other biological fluids or tissues.

By the term “normal cell” (control sample) is meant herein a cell sample that does not contain a specifically chosen cancer. Control samples can be obtained from patients/individuals not afflicted with cancer. Alternatively, a control sample can be taken from a non-afflicted tissue of a suspected cancer patient. Other types of control samples may also be used, such as a non-tumor cell line.

In an embodiment, the above-mentioned prevention/treatment comprises the use/administration of more than one (i.e. a combination of) active agent (e.g., one or more compounds of formula I, or salts thereof). The combination of prophylactic/therapeutic agents and/or compositions of the present invention may be administered or co-administered (e.g., consecutively, simultaneously, at different times) in any conventional dosage form. Co-administration in the context of the present invention refers to the administration of more than one therapeutic in the course of a coordinated treatment to achieve an improved clinical outcome. Such co-administration may also be coextensive, that is, occurring during overlapping periods of time. For example, a first agent may be administered to a patient before, concomitantly, before and after, or after a second active agent is administered. The agents may in an embodiment be combined/formulated in a single composition and thus administered at the same time. In an embodiment, the one or more active agent(s) of the present invention is used/administered in combination with one or more agent(s) currently used to prevent or treat cancer (e.g., carcinoma), including chemotherapeutical agents, such as Cisplatin™, Oxaliplatin™ and their derivatives, nucleotide analogues (e.g., 5-fluorouracyl), kinase inhibitors etc.

The invention further provides kits or packages (e.g., commercial kits or packages) comprising the above-mentioned compound(s), composition(s) or agent(s). The kits may also comprise instructions for the use of the compound(s), composition(s) or agent(s) for the prevention or treatment of cancer (e.g., carcinoma) in a subject. The kit or package may further comprise other components, such as buffers, containers and/or devices for administering the agent(s)/composition(s) to a subject (e.g., syringe and/or vial and/or ampoule).

In another aspect, the present invention provides a method of preparing the above-mentioned compound of formula (I) or a salt thereof.

A method for preparing a compound of formula (I):

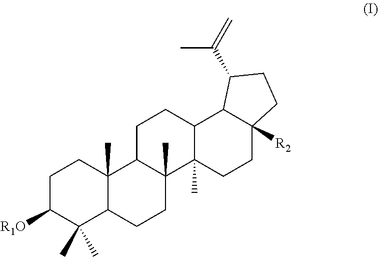

wherein R₁ is a sugar moiety, and R₂ is CH₂O-sugar moiety or COO-sugar moiety; said method comprising

(i) (a) glycosylating the C-28 position of betulin 3-acetate with a perbenzoylated or peracetylated trichloroacetimidate or trifluorophenylacetimidate sugar donor under the promotion of a Lewis acid to yield a first glycosylated compound; (b) submitting the first glycosylated compound to regioselective deacetylation conditions to cleave the acetyl group at the C-3 position to yield a deacetylated compound; (c) glycosylating the C-3 position of the deacetylated compound with a perbenzoylated or peracetylated trichloroacetimidate or trifluorophenylacetimidate sugar donor under the promotion of a Lewis acid to yield a second glycosylated compound; and (d) submitting the second glycosylated compound to deacetylation conditions, or

(ii) (a) glycosylating the C-28 position of betulinic acid with a perbenzoylated or peracetylated bromide sugar donor under phase-transfer conditions to yield a first glycosylated compound; (b) glycosylating the C-3 position of the first glycosylated compound with a perbenzoylated or peracetylated trichloroacetimidate or trifluorophenylacetimidate sugar donor under the promotion of a Lewis acid to yield a second glycosylated compound; and (c) submitting the second glycosylated compound to deacetylation conditions, or

(iii) (a) glycosylating the C-3 and C-28 positions of betulin or betulinic acid with a perbenzoylated or peracetylated trichloroacetimidate or trifluorophenylacetimidate sugar donor under the promotion of a Lewis acid via a Schmidt's inverse procedure to yield a glycosylated compound; and (b) submitting the glycosylated compound to deacetylation conditions.

In an embodiment, the above-mentioned sugar moiety is D-glucose, D-galactose, D-mannose, D-glucuronic acid, D-galacturonic acid, L-rhamnose, L-arabinose, D-arabinose, L-fucose, D-fucose, D-xylose, D-lyxose, D-allose, D-gulose, D-idose, D-talose, D-apiose, D-lactose, D-maltose, D-cellobiose, D-maltotriose, or D-maltotetraose.

In another aspect, the present invention provides a method for preparing a compound of formula (I):

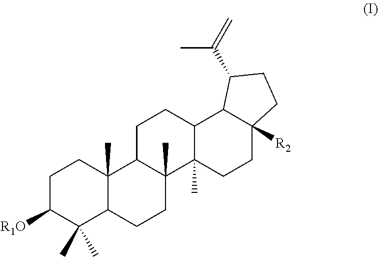

wherein R₁ is α-L-arabinopyranose or α-L-rhamnopyranose, and R₂ is CH₂O-8-D-glucopyranose; said method comprising (a) glycosylating the C-28 position of betulin 3-acetate with a perbenzoylated or peracetylated trichloroacetimidate or trifluorophenylacetimidate glucose donor under the promotion of a Lewis acid to yield a first glycosylated compound; (b) submitting the first glycosylated compound to regioselective deacetylation conditions to cleave the acetyl group at the C-3 position to yield a deacetylated compound; (c) glycosylating the C-3 position of the deacetylated compound with a perbenzoylated or peracetylated trichloroacetimidate or trifluorophenylacetimidate arabinose or rhamnose donor under the promotion of a Lewis acid to yield a second glycosylated compound; and (d) submitting the second glycosylated compound to deacetylation conditions.

In an embodiment, the above-mentioned Lewis acid of (a) is (i) trimethylsilyl trifluoromethanesulfonate (TMSOTf), (ii) Pert-butyldimethylsilyl trifluoromethanesulfonate (TBSOTf), (iii) boron trifluoride diethyletherate (BF₃-OEt₂), or (iv) any combination of (i) to (iii).

In another embodiment, the above-mentioned Lewis acid of (c) is (i) trimethylsilyl trifluoromethanesulfonate (TMSOTf), (ii) Pert-butyldimethylsilyl trifluoromethanesulfonate (TBSOTf), (iii) boron trifluoride diethyletherate (BF₃-OEt₂), or (iv) any combination of (i) to (iii).

In an embodiment, the above-mentioned regioselective deacetylation conditions comprise (a) acetyl chloride (AcCl) in a solution of CH₂Cl₂/MeOH, (b) para-toluenesulfonic acid monohydrate (TsOH.H₂O) in a solution of CH₂Cl₂/MeOH at 40° C., or (c) hydrazine hydrate (NH₂NH₂.x H₂O) in tetrahydrofuran (THF).

In an embodiment, the above-mentioned deacetylation conditions of (d) comprise (i) NaOMe and MeOH (Zemplén deacetylation conditions) or (ii) NaOH in MeOH/tetrahydrofuran/H₂O.

In an embodiment, the above-mentioned perbenzoylated or peracetylated trichloroacetimidate or trifluorophenylacetimidate glucose donor is 2,3,4,6-tetra-O-benzoyl-α-D-glucopyranosyl trichloroacetimidate.

In an embodiment, the above-mentioned perbenzoylated or peracetylated trichloroacetimidate or trifluorophenylacetimidate arabinose donor is 2,3,4-tri-O-benzoyl-β-L-arabinopyranosyl trichloroacetimidate.

In an embodiment, the above-mentioned perbenzoylated or peracetylated trichloroacetimidate or trifluorophenylacetimidate rhamnose donor is 2,3,4-tri-O-benzoyl-α-L-rhamnopyranosyl trichloroacetimidate.

In another aspect, the present invention provides a method for preparing a compound of formula (I):

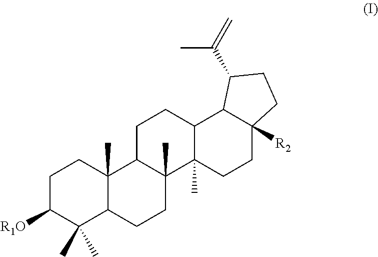

wherein R₁ is α-L-arabinopyranose or α-L-rhamnopyranose, and R₂ is COO-β-D-glucopyranose; said method comprising (a) glycosylating the C-28 position of betulinic acid with a perbenzoylated or peracetylated bromide glucose donor under phase-transfer conditions to yield a first glycosylated compound; (b) glycosylating the C-3 position of the first glycosylated compound with a perbenzoylated or peracetylated trichloroacetimidate or trifluorophenylacetimidate rhamnose or arabinose donor under the promotion of a Lewis acid to yield a second glycosylated compound; and (c) submitting the second glycosylated compound to deacetylation conditions.

In an embodiment, the above-mentioned deacetylation conditions comprises (i) NaOMe and MeOH (Zemplén deacetylation conditions) or (ii) NaOH in MeOH/tetrahydrofuran/H₂O.

In an embodiment, the above-mentioned NaOH is at about 0.5 N.

In an embodiment, the above-mentioned phase-transfer conditions comprises K₂CO₃, a quaternary ammonium salt, CH₂Cl₂/H₂O and reflux.

In a further embodiment, the above-mentioned quaternary ammonium salt is Bu₄NI, Bu₄NBr, Bu₄NCl, Aliquat™ 100, Aliquat™ 175, Aliquat™ 336 or Aliquat™ HTA-1.

In an embodiment, the above-mentioned perbenzoylated or peracetylated bromide glucose donor is 2,3,4,6-tetra-O-benzoyl-α-D-glucopyranosyl bromide.

In an embodiment, the above-mentioned perbenzoylated or peracetylated trichloroacetimidate or trifluorophenylacetimidate arabinose donor is 2,3,4-tri-O-benzoyl-β-L-arabinopyranosyl trichloroacetimidate.

In an embodiment, the above-mentioned perbenzoylated or peracetylated trichloroacetimidate or trifluorophenylacetimidate rhamnose donor is 2,3,4-tri-O-benzoyl-α-L-rhamnopyranosyl trichloroacetimidate.

In another aspect, the present invention provides a method for preparing a compound of formula (I):

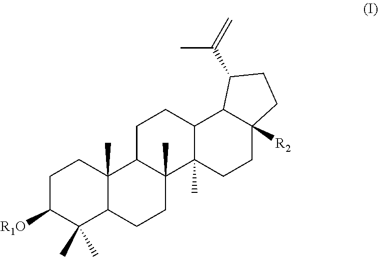

wherein when R₁ is β-D-glucopyranose, R₂ is COO-β-D-glucopyranose or CH₂O-β-D-glucopyranose; when R₁ is α-L-rhamnopyranose, R₂ is COO-β-L-rhamnopyranose or CH₂O-β-L-rhamnopyranose; said method comprising (a) glycosylating the C-3 and C-28 positions of betulin or betulinic acid with a perbenzoylated or peracetylated trichloroacetimidate or trifluorophenylacetimidate glucose or rhamnose donor under the promotion of a Lewis acid via a Schmidt's inverse procedure to yield a glycosylated compound; and (b) submitting the glycosylated compound to deacetylation conditions.

In an embodiment, the above-mentioned Lewis acid is (i) trimethylsilyl trifluoromethanesulfonate (TMSOTf), (ii) Pert-butyldimethylsilyl trifluoromethanesulfonate (TBSOTf), (iii) boron trifluoride diethyletherate (BF₃-OEt₂), or (iv) any combination of (i) to (iii).

In an embodiment, the above-mentioned deacetylation conditions comprises (i) NaOMe and MeOH (Zemplén deacetylation conditions) or (ii) NaOH in MeOH/tetrahydrofuran/H₂O.

In an embodiment, the above-mentioned step (a) glycosylates the C-3 and C-28 positions of betulin.

In another embodiment, the above-mentioned step (a) glycosylates the C-3 and C-28 positions of betulinic acid.

In an embodiment, the above-mentioned perbenzoylated or peracetylated trichloroacetimidate or trifluorophenylacetimidate glucose donor is 2,3,4,6-tetra-O-benzoyl-α-D-glucopyranosyl trichloroacetimidate.

In an embodiment, the above-mentioned perbenzoylated or peracetylated trichloroacetimidate or trifluorophenylacetimidate rhamnose donor is 2,3,4-tri-O-α-L-rhamnopyranosyl trichloroacetimidate.

In an embodiment, the above-mentioned Schmidt's inverse procedure comprises pre-mixing said betulin or betulinic acid with said Lewis acid before adding said perbenzoylated or peracetylated trichloroacetimidate or trifluorophenylacetimidate glucose or rhamnose donor.

In a further embodiment, the above-mentioned addition of the perbenzoylated or peracetylated trichloroacetimidate or trifluorophenylacetimidate glucose or rhamnose donor is performed at a temperature of between about −78° C. to about 25° C. In a further embodiment, the above-mentioned addition of the perbenzoylated or peracetylated trichloroacetimidate or trifluorophenylacetimidate glucose or rhamnose donor is performed at a temperature of about −10° C.

In an embodiment, the above-mentioned method for preparing a compound of formula I is a method described in Example 1 below.

Although various embodiments of the invention are disclosed herein, many adaptations and modifications may be made within the scope of the invention in accordance with the common general knowledge of those skilled in this art. Such modifications include the substitution of known equivalents for any aspect of the invention in order to achieve the same result in substantially the same way. Numeric ranges are inclusive of the numbers defining the range. In the claims, the word “comprising” is used as an open-ended term, substantially equivalent to the phrase “including, but not limited to”. The articles “a,” “an” and “the” are used herein to refer to one or to more than one (i.e., to at least one) of the grammatical object of the article.

The present invention is illustrated in further details by the following non-limiting examples, which are illustrative of various aspects of the invention, and do not limit the broad aspects of the invention as disclosed herein.

### EXAMPLES

### Example 1

### Materials and Methods

### Chemicals

Chemical reagents were purchased from Sigma-Aldrich Co. Canada or Alfa Aesar Co. and were used as received. The usual solvents were obtained from VWR International Co. and were used as received. Air and water sensitive reactions were performed in flame-dried glassware under an argon atmosphere. Moisture sensitive reagents were introduced via a dry syringe. Dichloromethane (CH₂Cl₂) and acetone were distilled from anhydrous CaH₂ under an argon atmosphere. Tetrahydrofuran (THF) was distilled from sodium/benzophenone ketyl under an argon atmosphere. Methanol (MeOH) was distilled from Mg and I₂ under an argon atmosphere. Analytical thin-layer chromatography was performed with silica gel 60 F₂₅₄, 0.25 mm pre-coated TLC plates (Silicycle, Québec, Canada). Compounds were visualized using UV₂₅₄ and cerium molybdate (2 g Ce(SO₄)₄(NH₄)₄, 5 g MoO₄(NH₄)₂, 200 mL H₂O, 20 mL H₂SO₄) with charring. Flash column chromatography was carried out using 60-230 mesh silica gel (Silicycle, Québec, Canada). All of the chemical yields were unoptimized and generally represent the highest result obtained for three independent experiments. Nuclear magnetic resonance (NMR) spectra were recorded on a Bruker Avance™ spectrometer at 400 MHz (¹H) and 100 MHz (¹³C), equipped with a 5 mm QNP probe. Elucidations of chemical structures were based on ¹H, ¹³C, COSY, TOCSY, HMBC, HSQC, J-resolved and DEPT-135 experiments. Signals were reported as m (multiplet), s (singlet), d (doublet), t (triplet), dd (doublet of doublet), ddt (doublet of doublet of triplet), br s (broad singlet) and coupling constants are reported in hertz (Hz). The chemical shifts were reported in ppm (δ) relative to residual solvent peak. The labile OH NMR signals appearing sometimes were not listed. Optical rotations were obtained using sodium D line at ambient temperature on a Rudolph Research Analytical Autopol™ IV automatic polarimeter. High-resolution electrospray ionization mass spectra (HR-ESI-MS) were obtained at the Department of Chemistry, Université de Montreal, Québec, Canada. Sugar donor 2,3,4,6-tetra-O-benzoyl-α-D-glucopyranose trifluorophenylacetimidate (5) (Yu, B. and Tao, H.; Tetrahedron Lett. 2001, 42: 2405-2407) was synthesized from D-glucose. Betulin (1) was extracted from the outer bark of Betula papyrifera March, and recrystallized with an azeotropic mixture of 2-butanol/H₂O (37:13) to afford crude 1 with a purity of >95% according to GC-MS. Betulinic acid (2) was purchased from Indofine Chemical Company Inc. 28-O-β-D-Glucopyranosyl betulin (Gauthier, C. of al., Bioorg. Med. Chem. 2006, 14: 6713-6725), 28-O-β-D-glucopyranosyl betulinic acid (Baglin, I. et al., J. Enzym. Inhib. Med. Ch. 2003, 18: 111-117), 28-O-tert-butyldiphenylsilyl betulin (9) and betulin 3-acetate (13) (Thibeault, D. et al., Bioorg. Med. Chem. 2007, 15: 6144-6157) were synthesized according to previously reported procedures.

### Synthesis of 28-O-tert-Butyldiphenylsilyi betulin 3β-O-2,3,4-tri-O-benzoyl-α-L-arabinopyranoside (Compound 10a)

The acceptor 9 (750 mg, 1.10 mmol) and the donor 7 (1.00 g, 1.65 mmol) were stirred at room temperature in anhydrous CH₂Cl₂ (16.5 mL, 15 mL·mmol⁻¹) with 4 Å MS under an argon atmosphere during 60 min. Then, the promoter TMSOTf (12 μL, 0.055 mmol) was injected in the medium via a dry syringe while keeping rigorous anhydrous conditions. The mixture was stirred 2.5 h at room temperature and quenched by addition of Et₃N (0.61 mL, 4.4 mmol). The solvents were evaporated under reduced pressure, then the resulting oily residue was purified by flash chromatography (hexanes/Et₂O 9:1 to 17:3) to afford 10a (874 mg, 71%) as a white crystalline powder. R_{f} 0.67 (hexanes/EtOAc 3:1); [α]²⁵_{D} +71.0° (c 1.0, CHCl₃); ¹H NMR (CDCl₃, 400 MHz) δ: 8.08-7.27 (25H, aromatic protons), 5.78 (1H, dd, J=8.7, 6.5 Hz, H-2′), 5.68 (1H, m, H-4′), 5.60 (1H, dd, J=8.9, 3.5 Hz, H-3′), 4.78 (1H, d, J=6.4 Hz, H-1′), 4.59 (1H, br s, H-29), 4.52 (1H, br s, H-29), 4.32 (1H, dd, J=13.0, 3.8 Hz, H-5′), 3.86 (1H, dd, J=12.9, 1.8 Hz, H-5′), 3.68 (1H, d, J=9.9 Hz, H-28), 3.32 (1H, d, J=10.0 Hz, H-28), 3.13 (1H, dd, J=11.4, 4.8 Hz, H-3), 2.26 (1H, td, J=11.0, 5.6 Hz, H-19), 1.64 (3H, s, H-30), 1.06 (9H, s, C(CH₃)₃), 0.91 (3H, s, H-27), 0.77 (3H, s, H-23), 0.75 (3H, s, H-25), 0.68 (3H, s, H-26), 0.64 (3H, s, H-24). ¹³CNMR (CDCl₃, 100 MHz) δ: 165.8-165.2 (3×CO), 150.7 (C-20), 135.7-127.6 (aromatic carbons), 109.4 (C-29), 103.0 (C-1′), 90.1 (C-3), 70.8 (C-3′), 70.2 (C-2′), 68.7 (C-4′), 62.6 (C-5′), 61.0 (C-28), 55.5 (C-5), 50.3 (C-9), 48.4 (C-18), 48.4 (C-17), 47.8 (C-19), 42.6 (C-14), 40.7 (C-8), 39.0 (C-4), 38.6 (C-1), 37.2 (C-13), 36.8 (C-10), 34.5 (C-22), 34.1 (C-7), 29.8 (C-21), 29.5 (C-16), 27.7 (C-23), 27.0 (C-15), 26.9 (C(CH₃)₃), 26.1 (C-2), 25.1 (C-12), 20.7 (C-11), 19.4 (C(CH₃)₃), 19.1 (C-30), 18.1 (C-6), 16.0 (C-24), 16.0 (C-25), 15.7 (C-26), 14.6 (C-27). HR-ESI-MS m/z 1147.6111 [M+Na]⁺ (calcd for C₇₂H₈₈O₉SiNa, 1147.6090).

### Synthesis of 28-O-tert-Butyldiphenylsilyl betulin 3β-O-2,3,4-tri-O-benzoyl-α-L-rhamnopyranoside (Compound 10b)

This compound was prepared from the acceptor 9 (500 mg, 0.734 mmol) and the donor 8 (684 mg, 1.10 mmol) in the same manner as that described for compound 10a. Purification by flash chromatography (isocratic hexanes/Et₂O 9:1) gave 10b (634 mg, 76%) as a white crystalline powder. R_{f} 0.77 (hexanes/EtOAc 3:1); [α]²⁵_{D} +46.6° (c 0.5, CHCl₃); ¹H NMR (CDCl₃, 400 MHz) δ: 8.13-7.21 (25H, aromatic protons), 5.84 (1H, dd, J=10.1, 3.3 Hz, H-3′), 5.68 (1H, m, H-4′), 5.65 (1H, m, H-2′), 5.08 (1H, d, J=1.1 Hz, H-1′), 4.60 (1H, d, J=1.8 Hz, H-29), 4.53 (1H, br s, H-29), 4.30 (1H, m, H-5′), 3.70 (1H, d, J=9.9 Hz, H-28), 3.34 (1H, d, J=9.9 Hz, H-28), 3.20 (1H, t, J=8.3 Hz, H-3), 2.27 (1H, td, J=10.8, 5.6 Hz, H-19), 1.65 (3H, s, H-30), 1.33 (3H, d, J=6.2 Hz, H-6′), 1.07 (9H, s, C(CH₃)₃), 1.06 (3H, s, H-23), 0.94 (3H, s, H-24), 0.94 (3H, s, H-27), 0.83 (3H, s, H-25), 0.72 (3H, s, H-26). ¹³C NMR (CDCl₃, 100 MHz) δ: 165.8-165.6 (3×CO), 150.8 (C-20), 135.7-127.6 (aromatic carbons), 109.4 (C-29), 99.7 (C-1′), 90.0 (C-3), 72.0 (C-4′), 71.2 (C-2′), 70.2 (C-3′), 66.8 (C-5′), 61.1 (C-28), 55.4 (C-5), 50.3 (C-9), 48.4 (C-18), 48.4 (C-17), 47.8 (C-19), 42.6 (C-14), 40.8 (C-8), 39.1 (C-4), 38.6 (C-1), 37.2 (C-13), 36.9 (C-10), 34.5 (C-22), 34.1 (C-7), 29.9 (C-21), 29.5 (C-16), 28.3 (C-23), 27.0 (C-15), 26.9 (C(CH₃)₃), 25.6 (C-2), 25.1 (C-12), 20.8 (C-11), 19.4 (C(CH₃)₃), 19.1 (C-30), 18.3 (C-6), 17.6 (C-6′), 16.4 (C-24), 16.1 (C-25), 15.7 (C-27), 14.7 (C-26). HR-ESI-MS m/z 1161.6262 [M+Na]⁺ (calcd for C₇₃H₉₀O₉SiNa, 1161.6252).

### Synthesis of betulin 38-O-2,3,4-tri-O-benzoyl-α-L-arabinopyranoside (Compound 11a)

To a solution of 10a (200 mg, 0.178 mmol) in anhydrous THF (1.94 mL) was added HOAc (224 μL, 3.91 mmol) and 1 M TBAF in THF (3.88 mL) at room temperature under an argon atmosphere. The mixture was refluxed overnight or until TLC (hexanes/EtOAc 4:1) showed the disappearance of the initial product. Then, the mixture was diluted with EtOAc, washed with H₂O, dried over anhydrous MgSO₄, filtered and the solvents were evaporated under reduced pressure. The resulting residue was purified by flash chromatography (hexanes/Et₂O 9:1 to 3:2) to furnish 11a (117 mg, 75%) as a white amorphous solid. R_{f} 0.27 (hexanes/EtOAc 3:1); [α]²⁵_{D} +103.6° (c 0.1, CHCl₃); ¹H NMR (CDCl₃, 400 MHz) δ: 8.09-7.27 (15H, aromatic protons), 5.77 (1H, dd, J=8.9, 6.5 Hz, H-2′), 5.67 (1H, m, H-4′), 5.60 (1H, dd, J=8.9, 3.5 Hz, H-3′), 4.78 (1H, d, J=6.5 Hz, H-1′), 4.68 (1H, d, J=1.8 Hz, H-29), 4.57 (1H, br s, H-29), 4.33 (1H, dd, J=13.0, 3.8 Hz, H-5′), 3.88 (1H, dd, J=12.9, 1.9 Hz, H-5′), 3.78 (1H, d, J=10.7 Hz, H-28), 3.32 (1H, d, J=10.7 Hz, H-28), 3.14 (1H, dd, J=11.3, 4.8 Hz, H-3), 2.38 (1H, td, J=10.7, 5.6 Hz, H-19), 1.68 (3H, s, H-30), 0.98 (3H, s, H-26), 0.95 (3H, s, H-27), 0.80 (3H, s, H-25), 0.76 (3H, s, H-23), 0.64 (3H, s, H-24). ¹³C NMR (CDCl₃, 100 MHz) δ: 165.8-165.2 (3×CO), 150.4 (C-20), 133.3-128.3 (aromatic carbons), 109.7 (C-29), 103.0 (C-1′), 90.1 (C-3), 70.7 (C-3′), 70.2 (C-2′), 68.7 (C-4′), 62.6 (C-5′), 60.4 (C-28), 55.5 (C-5), 50.3 (C-9), 48.7 (C-18), 47.7 (C-17), 47.7 (C-19), 42.6 (C-14), 40.9 (C-8), 39.0 (C-4), 38.7 (C-1), 37.2 (C-13), 36.8 (C-10), 34.1 (C-7), 33.9 (C-22), 29.7 (C-21), 29.1 (C-16), 27.7 (C-23), 27.0 (C-15), 26.1 (C-2), 25.2 (C-12), 20.8 (C-11), 19.1 (C-30), 18.1 (C-6), 16.0 (C-25), 16.0 (C-24), 15.9 (C-26), 14.7 (C-27). HR-ESI-MS m/z 909.4957 [M+Na]⁺ (calcd for C₅₆H₇₀O₉Na, 909.4912).

### Synthesis of betulin 30-O-2,3,4-tri-O-benzoyl-α-L-rhamnopyranoside (Compound 11b)

This compound was prepared from 10b (200 mg, 0.176 mmol) in the same manner as that described for compound 11a. Purification by flash chromatography (hexanes/EtOAc 9:1 to 3:2) gave 11b (138 mg, 87%) as a white crystalline powder. R_{f} 0.33 (hexanes/EtOAc 3:1); [α]²⁵_{D} +76.6° (c 1.0, CHCl₃); ¹H NMR (CDCl₃, 400 MHz) δ: 8.13-7.23 (15H, aromatic protons), 5.82 (1H, dd, J=10.2, 3.3 Hz, H-3′), 5.67 (1H, t, J=10.0 Hz, H-4′), 5.64 (1H, dd, J=3.3, 1.8 Hz, H-2′), 5.08 (1H, d, J=1.4 Hz, H-1′), 4.69 (1H, d, J=2.1 Hz, H-29), 4.58 (1H, br s, H-29), 4.30 (1H, ddt, J=9.7, 6.2, 6.2 Hz, H-5′), 3.81 (1H, d, J=10.8 Hz, H-28), 3.34 (1H, d, J=10.8 Hz, H-28), 3.20 (1H, dd, J=8.7, 7.5 Hz, H-3), 2.39 (1H, td, J=10.5, 5.6 Hz, H-19), 1.68 (3H, s, H-30), 1.33 (3H, d, J=6.2 Hz, H-6′), 1.05 (3H, s, H-23), 1.04 (3H, s, H-26), 0.98 (3H, s, H-27), 0.93 (3H, s, H-24), 0.89 (3H, s, H-25). ¹³C NMR (CDCl₃, 100 MHz) δ: 165.9-165.6 (3×CO), 150.5 (C-20), 133.4-128.3 (aromatic carbons), 109.7 (C-29), 99.7 (C-1′), 90.0 (C-3), 72.0 (C-4′), 71.2 (C-2′), 70.2 (C-3′), 66.8 (C-5′), 60.6 (C-28), 55.5 (C-5), 50.4 (C-9), 48.8 (C-18), 47.8 (C-19), 47.8 (C-17), 42.7 (C-14), 41.0 (C-8), 39.2 (C-4), 38.7 (C-1), 37.3 (C-13), 36.9 (C-10), 34.2 (C-7), 34.0 (C-22), 29.8 (C-21), 29.2 (C-16), 28.3 (C-23), 27.0 (C-15), 25.7 (C-2), 25.2 (C-12), 20.9 (C-11), 19.1 (C-30), 18.3 (C-6), 17.6 (C-6), 16.4 (C-24), 16.2 (C-25).

### Synthesis of 28-O-2,3,4,6-Tetra-O-benzoyl-β-D-glucopyranosyl betulin 3-acetate (Compound 14)

This compound was prepared from the acceptor 13 (700 mg, 1.44 mmol) and the donor 4 (1.61 g, 2.17 mmol) in the same manner as that described for compound 10a except for the molar volume of CH₂Cl₂ (40 mL·mmol⁻¹). Purification by flash chromatography (hexanes/EtOAc 4:1 to 7:3) gave 14 (903 mg, 60%) as a white foam. R_{f} 0.49 (hexanes/EtOAc 3:1); [α]²⁵_{D}+24.7° (c 0.2, CHCl₃); ¹H NMR (CDCl₃, 400 MHz) δ: 8.06-7.26 (20H, aromatic protons), 5.93 (1H, t, J=9.7 Hz, H-3″), 5.67 (1H, t, J=9.7 Hz, H-4″), 5.56 (1H, dd, J=9.8, 8.0 Hz, H-2″), 4.79 (1H, d, J=8.0 Hz, H-1″), 4.65 (1H, m, H-6″), 4.63 (1H, m, H-29), 4.55 (1H, m, H-29), 4.53 (1H, m, H-6″), 4.45 (1H, m, H-3), 4.17 (1H, ddd, J=9.4, 5.6, 3.3 Hz, H-5″), 3.67 (1H, d, J=8.9 Hz, H-28), 3.58 (1H, d, J=8.9 Hz, H-28), 2.28 (1H, m, H-19), 2.05 (3H, s, CH₃CO), 1.63 (3H, s, H-30), 0.84 (3H, s, H-23), 0.84 (3H, s, H-24), 0.83 (3H, s, H-26), 0.82 (3H, s, H-27), 0.80 (3H, s, H-25). ¹³C NMR (CDCl₃, 100 MHz) δ: 171.1 (CH₃CO), 166.2-165.3 (4×CO), 150.4 (C-20), 133.5-128.3 (aromatic carbons), 109.7 (C-29), 102.1 (C-1″), 80.9 (C-3), 72.9 (C-3″), 72.2 (C-5″), 71.8 (C-2″), 70.1 (C-4″), 68.9 (C-28), 63.3 (C-6″), 55.3 (C-5), 50.2 (C-9), 48.6 (C-18), 48.0 (C-19), 47.0 (C-17), 42.5 (C-14), 40.7 (C-8), 38.3 (C-1), 37.8 (C-4), 37.6 (C-13), 37.0 (C-10), 34.7 (C-22), 33.8 (C-7), 29.6 (C-21), 29.2 (C-16), 28.0 (C-23), 27.0 (C-15), 25.0 (C-12), 23.7 (C-2), 21.4 (CH₃CO), 20.8 (C-11), 19.0 (C-30), 18.1 (C-6), 16.5 (C-24), 16.2 (C-25), 15.8 (C-26), 14.7 (C-27). HR-ESI-MS m/z 1085.5384 [M+Na]⁺ (calcd for C₆₆H₇₈O₁₂Na, 1085.5386).

### Synthesis of 28-O-2,3,4,6-Tetra-O-benzoyl-β-D-glucopyranosyl betulin (Compound 15)

To a solution of 14 (840 mg, 0.790 mmol) in anhydrous CH₂Cl₂/MeOH 1:2 (60 mL) was added AcCl (1.19 mL, 16.8 mmol) at 0° C. (ice/water bath). The mixture was stirred overnight at room temperature or until TLC (hexanes/EtOAc 7:3) showed the disappearance of the initial product. Then, the reaction was quenched with Et₃N (4.68 mL, 33.6 mmol) and the solvents were evaporated under reduced pressure to give a residue which was purified by flash chromatography (hexanes/EtOAc 4:1 to 3:2) to afford 15 (523 mg, 75%, corrected yield) as a white crystalline powder along with 14 (87 mg, 10%, recovery yield) as a white foam. R_{f} 0.20 (hexanes/EtOAc 3:1); [α]²⁵_{D} +27.0° (c 0.5, CHCl₃); ¹H NMR (CDCl₃, 400 MHz) δ: 8.05-7.25 (20H, aromatic protons), 5.93 (1H, t, J=9.7 Hz, H-3″), 5.67 (1H, t, J=9.7 Hz, H-4″), 5.56 (1H, dd, J=9.7, 7.8 Hz, H-2″), 4.79 (1H, d, J=8.0 Hz, H-1″), 4.64 (1H, m, H-6″), 4.63 (1H, m, H-29), 4.54 (1H, m, H-29), 4.53 (1H, m, H-6″), 4.17 (1H, m, H-5″), 3.66 (1H, d, J=8.9 Hz, H-28), 3.58 (1H, d, J=8.9 Hz, H-28), 3.17 (1H, dd, J=11.0, 4.6 Hz, H-3), 2.27 (1H, m, H-19), 1.63 (3H, s, H-30), 0.96 (3H, s, H-23), 0.83 (3H, s, H-26), 0.83 (3H, s, H-27), 017 (3H, s, H-25), 0.76 (3H, s, H-24). ¹³C NMR (CDCl₃, 100 MHz) δ: 166.1-165.0 (4×CO), 150.4 (C-20), 133.4-128.3 (aromatic carbons), 109.6 (C-29), 102.0 (C-1″), 78.9 (C-3), 72.8 (C-3″), 72.2 (C-5″), 71.7 (C-2″), 70.0 (C-4″), 68.8 (C-28), 63.3 (C-6″), 55.2 (C-5), 50.3 (C-9), 48.6 (C-18), 48.0 (C-19), 46.9 (C-17), 42.5 (C-14), 40.7 (C-8), 38.8 (C-4), 38.6 (C-1), 37.6 (C-13), 37.1 (C-10), 343 (C-22), 33.8 (C-7), 29.6 (C-21), 29.2 (C-16), 28.0 (C-23), 27.3 (C-2), 27.0 (C-15), 25.0 (C-12), 20.8 (C-11), 19.0 (C-30), 18.1 (C-6), 16.1 (C-25), 15.7 (C-26), 15.4 (C-24), 14.8 (C-27). HR-ESI-MS m/z 1043.5295 [M+Na]⁺ (calcd for C₆₄H₇₆O₁₁Na, 1043.5280).

### Synthesis of 28-O-2,3,4,6-Tetra-O-benzoyl-β-D-glucopyranosyl betulin 3β-O-2,3,4-tri-O-benzoyl-α-L-arabinopyranoside (Compound 12a)

The acceptor 15 (150 mg, 0.147 mmol) and the donor 7 (134 mg, 0.220 mmol) were stirred at room temperature in anhydrous CH₂Cl₂ (2.9 mL) with 4 Å MS under an argon atmosphere during 60 min. The temperature was lowered to 0° C. with an ice/water bath, then a solution of TMSOTf in CH₂Cl₂ (100 μL, 150 mM) was injected in the medium via a dry syringe while keeping rigorous anhydrous conditions. The mixture was stirred 3 h at room temperature and quenched by addition of Et₃N (82 μL, 0.59 mmol). The solvents were evaporated under reduced pressure to give a residue which was purified by Nash chromatography (hexanes/EtOAc 9:1 to 3:2) to afford 12a (132 mg, 62%) as a white foam. R_{f} 0.26 (hexanes/EtOAc 3:1); [α]²⁵_{D} +80.3° (c 0.2, CHCl₃); ¹H NMR (CDCl₃, 400 MHz) δ: 8.10-7.25 (35H, aromatic protons), 5.94 (1H, t, J=9.7 Hz, H-3″), 5.78 (1H, dd, J=8.9, 6.7 Hz, H-2′), 5.68 (1H, m, H-4′), 5.67 (1H, m, H-4″), 5.60 (1H, dd, J=9.1, 3.5 Hz, H-3′), 5.56 (1H, dd, J=9.9, 8.0 Hz, H-2″), 4.79 (1H, m, H-1″), 4.78 (1H, m, H-1′), 4.65 (1H, m, H-6″), 4.63 (1H, br s, H-29), 4.55 (1H, br s, H-29), 4.53 (1H, m, H-6″), 4.33 (1H, dd, J=13.0, 3.5 Hz, H-5′), 4.17 (1H, ddd, J=9.5, 5.4, 3.3 Hz, H-5″), 3.87 (1H, dd, J=13.0, 1.9 Hz, H-5′), 3.66 (1H, d, J=8.8 Hz, H-28), 3.57 (1H, d, J=8.8 Hz, H-28), 312 (1H, dd, J=11.3, 4.6 Hz, H-3), 2.28 (1H, m, H-19), 1.62 (3H, s, H-30), 0.81 (3H, s, H-27), 0.79 (3H, s, H-26), 0.76 (3H, s, H-23), 0.76 (3H, s, H-25), 0.65 (3H, s, H-24), 0.58 (1H, d, J=10.8 Hz, H-5), 0.50 (1H, br d, J=13.5 Hz, H-15). ¹³C NMR (CDCl₃, 100 MHz) δ: 166.1-164.9 (7×CO), 150.3 (C-20), 133.4-128.3 (aromatic carbons), 109.6 (C-29), 103.0 (C-1′), 102.0 (C-1″), 90.0 (C-3), 72.8 (C-3″), 72.1 (C-5″), 71.7 (C-2″), 70.7 (C-3′), 70.2 (C-2′), 70.0 (C-4″), 68.9 (C-28), 68.7 (C-4′), 63.2 (C-6″), 62.6 (C-5′), 55.4 (C-5), 50.2 (C-9), 48.5 (C-18), 47.9 (C-19), 46.9 (C-17), 42.4 (C-14), 40.6 (C-8), 38.9 (C-4), 38.6 (C-1), 37.6 (C-13), 36.7 (C-10), 34.6 (C-22), 33.7 (C-7), 29.6 (C-21), 29.1 (C-16), 27.6 (C-23), 26.9 (C-15), 26.0 (C-2), 25.0 (C-12), 20.7 (C-11), 19.0 (C-30), 17.9 (C-6), 16.0 (C-24), 16.0 (C-25), 15.7 (C-26), 14.7 (C-27). HR-ESI-MS m/z 1487.6499 [M+Na]₊ (calcd for C₉₀H₉₆O₁₈Na, 1487.6489).

### Synthesis of 28-O-2,3,4,6-Tetra-O-benzoyl-β-D-glucopyranosyl betulin 3β-O-2,3,4-tri-O-benzoyl-α-L-rhamnopyranoside (Compound 12b)

This compound was prepared from the acceptor 15 (17 mg, 0.017 mmol) and the donor 8 (16 mg, 0.025 mmol) in the same manner as that described for compound 12a except for the concentration of the solution of TMSOTf in CH₂Cl₂ (20 mM). Purification by flash chromatography (hexanes/EtOAc 9:1 to 3:1) gave 12b (18 mg, 72%) as a white foam. R_{f} 0.34 (hexanes/EtOAc 3:1); [α]²⁵_{D}+57.1° (c 0.2, CHCl₃); ¹H NMR (CDCl₃, 400 MHz) δ: 8.15-7.24 (35H, aromatic protons), 5.95 (1H, t, J=9.7 Hz, H-3″), 5.83 (1H, dd, J=10.2, 3.3 Hz, H-3′), 5.68 (1H, m, H-4″), 5.68 (1H, m, H-4″), 5.65 (1H, m, H-2′), 5.56 (1H, dd, J=9.9, 8.0 Hz, H-2″), 5.07 (1H, d, J=1.3 Hz, H-1′), 4.80 (1H, d, J=8.1 Hz, H-1″), 4.66 (1H, m, H-6″), 4.63 (1H, m, H-29), 4.55 (1H, m, H-29), 4.54 (1H, m, H-6″), 4.32 (1H, dd, J=9.7, 6.0 Hz, H-5′), 4.18 (1H, ddd, J=9.4, 5.4, 3.3 Hz, H-5″), 3.67 (1H, d, J=9.1 Hz, H-28), 3.59 (1H, d, J=9.1 Hz, H-28), 3.18 (1H, t, J=8.1 Hz, H-3), 2.29 (1H, m, H-19), 1.63 (3H, s, H-30), 1.33 (3H, d, J=6.2 Hz, H-6), 1.04 (3H, s, H-23), 0.94 (3H, s, H-24), 0.85 (3H, s, H-26), 0.84 (3H, s, H-25), 0.83 (3H, s, H-27). ¹³C NMR (CDCl₃, 100 MHz) δ: 166.2-165.0 (7×CO), 150.4 (C-20), 133.4-128.3 (aromatic carbons), 109.6 (C-29), 102.1 (C-1″), 99.7 (C-1′), 90.0 (C-3), 72.8 (C-3″), 72.2 (C-5″), 72.0 (C-4″), 71.7 (C-2″), 71.2 (C-2′), 70.2 (C-3′), 70.0 (C-4), 68.9 (C-28), 66.8 (C-5′), 63.3 (C-6″), 55.4 (C-5), 50.2 (C-9), 48.6 (C-18), 48.0 (C-19), 46.9 (C-17), 42.5 (C-14), 40.7 (C-8), 39.1 (C-4), 38.6 (C-1), 37.6 (C-13), 36.8 (C-10), 34.7 (C-22), 33.8 (C-7), 29.6 (C-21), 29.2 (C-16), 28.2 (C-23), 26.9 (C-15), 25.6 (C-2), 25.0 (C-12), 20.8 (C-11), 19.0 (C-30), 18.1 (C-6), 17.6 (C-6′), 16.4 (C-24), 16.1 (C-25), 15.7 (C-26), 14.8 (C-27). HR-ESI-MS m/z 1501.6648 [M+Na]⁺ (calcd for C₉₁H₉₈O₁₈Na, 1501.6645).

### Synthesis of 28-O-β-D-Glucopyranosyl betulin 313-O-α-L-arabinopyranoside (Compound 16a)

To a solution of 12a (94 mg, 0.064 mmol) in MeOH/THF/H₂O 1:2:1 (4.4 mL) was added NaOH (52 mg, 1.3 mmol). The reaction mixture was stirred 5 h at room temperature or until TLC (CH₂Cl₂/MeOH 9:1) showed the complete disappearance of the benzoylated product and then acidified to pH≈4 with aqueous HCl 10%. The solvents were evaporated under reduced pressure to give a solid residue which was purified by C-18 reversed phase flash chromatography (MeOH/H₂O 4:1 to 9:1) to furnish 16a (40 mg, 86%) as a white amorphous powder. R_{f} 0.78 (CH₂Cl₂/MeOH 3:1); [α]²⁵_{D}-15.6° (c 0.1, MeOH); ¹H NMR (CDCl₃/CD₃OD 1:1, 400 MHz) δ: 4.68 (1H, d, J=1.6 Hz, H-29), 4.58 (1H, br s, H-29), 4.34 (1H, d, J=5.9 Hz, H-1′), 4.25 (1H, d, J=7.8 Hz, H-1″), 3.89 (1H, m, H-6″), 3.88 (1H, m, H-4′), 3.88 (1H, m, H-5′), 3.79 (1H, dd, J=11.9, 4.6 Hz, H-6″), 3.68 (1H, m, H-28), 3.65 (1H, m, H-2′), 3.61 (1H, m, H-3′), 3.61 (1H, m, H-28), 3.53 (1H, dd, J=13.8, 3.8 Hz, H-5′), 3.45 (1H, m, H-4″), 3.44 (1H, m, H-3″), 3.31 (1H, m, H-5″), 3.27 (1H, m, H-2″), 3.13 (1H, dd, J=11.3, 4.3 Hz, H-3), 2.43 (1H, td, J=10.3, 5.7 Hz, H-19), 1.69 (3H, s, H-30), 1.04 (3H, s, H-26), 1.01 (3H, s, H-23), 0.98 (3H, s, H-27), 0.84 (3H, s, H-25), 0.82 (3H, s, H-24), 0.73 (1H, d, J=10.3 Hz, H-5). ¹³C NMR: see Table I. HR-ESI-MS m/z 759.4635 [M+Na]⁺ (calcd for C₄₁H₆₈O₁₁Na, 759.4654).

### Synthesis of 28-O-β-D-Glucopyranosyl betulin 313-O-α-L-rhamnopyranoside (Compound 16b)

This compound was prepared from 12b (84 mg, 0.057 mmol) in the same manner as that described for compound 16a. Purification by C-18 reversed phase flash chromatography (MeOH/H₂O 4:1 to 100% MeOH) gave 16b (33 mg, 80%) as a white amorphous powder. R_{f} 0.78 (CH₂Cl₂/MeOH 3:1); [α]²⁵_{D} −42.8° (c 0.2, MeOH); ¹H NMR (CDCl₃/CD₃OD 1:1, 400 MHz) δ: 4.76 (1H, br s, H-1′), 4.68 (1H, br s, H-29), 4.58 (1H, br s, H-29), 4.25 (1H, d, J=7.8 Hz, H-1″), 3.90 (1H, m, H-6″), 3.89 (1H, m, H-2′), 3.78 (1H, m, H-6″), 3.75 (1H, m, H-5′), 3.70 (1H, m, H-28), 3.69 (1H, m, H-3′), 3.62 (1H, d, J=9.2 Hz, H-28), 3.43 (1H, m, H-4″), 3.42 (1H, m, H-3″), 3.39 (1H, m, H-4′), 3.31 (1H, m, H-5″), 3.27 (1H, m, H-2″), 3.08 (1H, dd, J=11.4, 4.6 Hz, H-3), 2.43 (1H, m, H-19), 2.09 (1H, br d, J=12.1 Hz, H-16), 1.69 (3H, s, H-30), 1.27 (3H, d, J=6.0 Hz, H-6′), 1.05 (3H, s, H-26), 0.99 (3H, s, H-27), 0.93 (3H, s, H-23), 0.85 (3H, s, H-25), 0.76 (3H, s, H-24). ¹³C NMR (CDCl₃/CD₃OD 1:1, 100 MHz) 5: see Table I. HR-ESI-MS m/z 773.4794 [M+Na]⁺ (calcd for C₄₂H₇₀O₁₁Na, 773.4810).

### Synthesis of 28-O-2,3,4,6-Tetra-O-benzoyl-β-D-glucopyranosyl betulinic acid (Compound 17)

To a solution of the acceptor 2 (500 mg, 1.10 mmol) and the donor 6 (939 mg, 1.42 mmol) in CH₂Cl₂ (12.7 mL) was added H₂O (12.7 mL), K₂CO₃ (378 mg, 2.74 mmol) and Bu₄NBr (141 mg, 0.438 mmol). The resulting mixture was vigorously stirred and refluxed for 6 h. Then, the mixture was diluted with CH₂Cl₂, washed with H₂O and brine. The solvents of the dried (MgSO₄) organic solution were evaporated under reduced pressure to give a brown residue which was purified by flash chromatography (100% CH₂Cl₂ to CH₂Cl₂/MeOH 49:1) to afford 17 (1.015 g, 90%) as a white crystalline powder. R_{f} 0.17 (hexanes/EtOAc 3:1); [α]²⁵_{D} +38.0° (c 0.5, CHCl₃); ¹H NMR (CDCl₃, 400 MHz) δ: 8.07-7.25 (20H, aromatic protons), 6.03 (1H, d, J=8.4 Hz, H-1″), 6.02 (1H, t, J=9.5 Hz, H-3″), 5.76 (1H, dd, J=9.9, 8.4 Hz, H-2″), 5.73 (1H, t, J=9.8 Hz, H-4″), 4.71 (1H, br s, H-29), 4.59 (1H, m, H-6″), 4.58 (1H, m, H-29), 4.48 (1H, dd, J=12.2, 5.6 Hz, H-6″), 4.29 (1H, ddd, J=9.5, 5.3, 2.9 Hz, H-5″), 3.13 (1H, dd, J=11.0, 4.6 Hz, H-3), 2.93 (1H, td, J=11.1, 4.8 Hz, H-19), 2.17 (1H, br d, J=13.2 Hz, H-16), 2.03 (1H, td, J=12.2, 3.2 Hz, H-13), 1.91 (1H, dd, J=12.7, 8.0 Hz, H-22), 1.63 (3H, s, H-30), 0.93 (3H, s, H-23), 0.79 (3H, s, H-27), 0.73 (3H, s, H-24), 0.68 (3H, s, H-25), 0.60 (1H, br d, J=14.3 Hz, H-15), 0.54 (1H, br d, J=10.5 Hz, H-5), 0.47 (3H, s, H-26), 0.38 (1H, br d, J=11.0 Hz, H-7). ¹³C NMR (CDCl₃, 100 MHz) δ: 174.0 (C-28), 166.1-164.7 (4×CO), 150.3 (C-20), 133.5-128.3 (aromatic carbons), 109.5 (C-29), 91.4 (C-1″), 78.9 (C-3), 73.0 (C-5″), 72.8 (C-3″), 70.3 (C-2″), 69.4 (C-4″), 62.7 (C-6″), 56.8 (C-17), 55.2 (C-5), 50.4 (C-9), 49.1 (C-18), 46.6 (C-19), 42.2 (C-14), 40.2 (C-8), 38.8 (C-4), 38.6 (C-1), 38.0 (C-13), 37.0 (C-10), 36.3 (C-22), 33.4 (C-7), 31.5 (C-16), 30.2 (C-21), 29.9 (C-15), 28.0 (C-23), 27.4 (C-2), 25.4 (C-12), 20.7 (C-11), 19.5 (C-30), 18.0 (C-6), 16.0 (C-25), 15.4 (C-26), 15.4 (C-24), 14.5 (C-27). HR-ESI-MS m/z 1057.5114 [M+Na]⁺ (calcd for C₆₄H₇₄O₁₂Na, 1057.5077).

### Synthesis of 28-O-2,3,4,6-Tetra-O-benzoyl-β-D-glucopyranosyl betulinic acid 3β-O-2,3,4-tri-O-benzoyl-α-L-arabinopyranoside (Compound 18a)

This compound was prepared from the acceptor 17 (250 mg, 0.241 mmol) and the donor 7 (220 mg, 0.362 mmol) in the same manner as that described for compound 10a except for the molar volume of CH₂Cl₂ (20 mL·mmol⁻¹). Purification by flash chromatography (hexanes/EtOAc 9:1 to 7:3) gave 18a (224 mg, 63%) as a white crystalline powder. R_{f} 0.22 (hexanes/EtOAc 3:1); [α]²⁵_{D} +88.9° (c 1.0, CHCl₃); ¹H NMR (CDCl₃, 400 MHz) δ: 8.09-7.23 (35H, aromatic protons), 6.04 (1H, m, H-1″), 6.03 (1H, m, H-3″), 5.78 (1H, m, H-2′), 5.76 (1H, m, H-2″), 5.75 (1H, m, H-4″), 5.68 (1H, m, H-4′), 5.61 (1H, m, H-3′), 4.77 (1H, d, H-1′), 4.71 (1H, m, H-29), 4.60 (1H, m, H-6″), 4.58 (1H, m, H-29), 4.50 (1H, m, H-6″), 4.32 (1H, m, H-5′), 4.30 (1H, m, H-5″), 3.87 (1H, m, H-5′), 3.09 (1H, m, H-3), 2.94 (1H, m, H-19), 1.64 (3H, s, H-30), 0.77 (3H, s, H-27), 0.74 (3H, s, H-23), 0.67 (3H, s, H-25), 0.62 (3H, s, H-24), 0.44 (3H, s, H-26). ¹³C NMR (CDCl₃, 100 MHz) δ: 174.0 (C-28), 166.0-164.7 (7×CO), 150.2 (C-20), 133.5-128.3 (aromatic carbons), 109.6 (C-29), 103.0 (C-1′), 91.4 (C-1″), 90.1 (C-3), 73.0 (C-5″), 72.8 (C-3″), 70.7 (C-3′), 70.2 (C-2″), 70.2 (C-2′), 69.4 (C-4″), 68.7 (C-4′), 62.7 (C-6″), 62.7 (C-5′), 56.8 (C-17), 55.4 (C-5), 50.3 (C-9), 49.1 (C-18), 46.6 (C-19), 42.1 (C-14), 40.2 (C-8), 38.9 (C-4), 38.6 (C-1), 38.0 (C-13), 36.7 (C-10), 36.3 (C-22), 33.3 (C-7), 31.5 (C-16), 30.2 (C-21), 29.8 (C-15), 27.7 (C-23), 26.0 (C-2), 25.4 (C-12), 20.7 (C-11), 19.5 (C-30), 17.8 (C-6), 16.1 (C-24), 15.9 (C-25), 15.3 (C-26), 14.4 (C-27). HR-ESI-MS m/z 1501.6347 [M+Na]⁺ (calcd for C₉₀H₉₄O₁₉Na, 1501.6282).

### Synthesis of 28-O-2,3,4,6-Tetra-O-benzoyl-β-D-glucopyranosyl betulinic acid 3β-O-2,3,4-tri-O-benzoyl-α-L-rhamnopyranoside (Compound 18b)

This compound was prepared from the acceptor 17 (250 mg, 0.241 mmol) and the donor 8 (225 mg, 0.362 mmol) in the same manner as that described for compound 10a except for the molar volume of CH₂Cl₂ (20 mL·mmol⁻¹). Purification by flash chromatography (hexanes/EtOAc 9:1 to 4:1) gave 18b (311 mg, 86%) as a white amorphous powder. R_{f} 0.33 (hexanes/EtOAc 3:1); [α]²⁵_{D} +72.5° (c 0.5, CHCl₃); ¹H NMR (CDCl₃, 400 MHz) δ: 8.10-7.21 (35H, aromatic protons), 6.08 (1H, m, H-1″), 6.07 (1H, m, H-3″), 5.83 (1H, m, H-3′), 5.82 (1H, m, H-2″), 5.77 (1H, m, H-4″), 5.69 (1H, m, H-4′), 5.67 (1H, m, H-2′), 5.08 (1H, br s, H-1′), 4.72 (1H, br s, H-29), 4.62 (1H, dd, J=12.3, 2.9 Hz, H-6″), 4.59 (1H, br s, H-29), 4.52 (1H, dd, J=12.3, 5.4 Hz, H-6″), 4.34 (1H, m, H-5″), 4.33 (1H, m, H-5′), 3.17 (1H, t, J=8.1 Hz, H-3), 2.96 (1H, td, J=10.8, 4.6 Hz, H-19), 2.20 (1H, br d, J=12.7 Hz, H-16), 1.64 (3H, s, H-30), 1.34 (3H, d, J=6.2 Hz, H-6′), 1.03 (3H, s, H-23), 0.92 (3H, s, H-24), 0.81 (3H, s, H-27), 0.77 (3H, s, H-25), 0.51 (3H, s, H-26), 0.44 (1H, br d, J=11.4 Hz, H-7). ¹³C NMR (CDCl₃, 100 MHz) δ: 174.0 (C-28), 166.0 (C—P), 163.5 (C—P), 150.1 (C-20), 133.6 (C—P), 128.2 (C—P), 109.5 (C-29), 99.7 (C-1′), 91.4 (C-1″), 90.0 (C-3), 72.9 (C-5″), 72.8 (C-3″), 71.9 (C-4′), 71.1 (C-2′), 70.2 (C-3′), 70.2 (C-2″), 69.3 (C-4″), 66.7 (C-5′), 62.7 (C-6″), 56.7 (C-17), 55.3 (C-5), 50.3 (C-9), 49.0 (C-18), 46.6 (C-19), 42.1 (C-14), 40.2 (C-8), 39.0 (C-4), 38.5 (C-1), 37.9 (C-13), 36.7 (C-10), 36.3 (C-22), 33.3 (C-7), 31.4 (C-16), 30.2 (C-21), 29.8 (C-15), 28.2 (C-23), 25.5 (C-2), 25.3 (C-12), 20.7 (C-11), 19.4 (C-30), 17.9 (C-6), 17.5 (C-6′), 16.3 (C-24), 16.0 (C-25), 15.3 (C-26), 14.4 (C-27). HR-ESI-MS m/z 1515.6419 [M+Na]⁺ (calcd for C₉₁H₉₆O₁₉Na, 1515.6438).

### Synthesis of 28-O-β-D-Glucopyranosyl betulinic acid 3β-O-α-L-arabinopyranoside (Compound 3)

This compound was prepared from 18a (100 mg, 0.068 mmol) in the same manner as that described for compound 16a. Purification by C-18 reversed phase flash chromatography (MeOH/H₂O 3:2 to 9:1) gave 3 (38 mg, 75%) as a white amorphous powder. R_{f} 0.78 (CH₂Cl₂/MeOH 3:1); [α]²⁵_{D} +12.2° (c 0.1, MeOH); ¹H NMR (CDCl₃/CD₃OD 1:2, 400 MHz) δ: 5.51 (1H, d, J=8.1 Hz, H-1″), 4.72 (1H, br s, H-29), 4.60 (1H, br s, H-29), 4.31 (1H, d, J=6.3 Hz, H-1′), 3.86 (1H, m, H-5′), 3.86 (1H, m, H-6″), 3.84 (1H, m, H-4′), 3.74 (1H, dd, J=12.1, 4.0 Hz, H-6″), 3.61 (1H, dd, J=8.4, 6.4 Hz, H-2′), 3.55 (1H, dd, J=8.4, 3.0 Hz, H-3′), 3.52 (1H, d, J=10.3 Hz, H-5′), 3.46 (1H, m, H-3″), 3.42 (1H, m, H-4″), 3.41 (1H, m, H-5″), 3.37 (1H, m, H-2″), 3.13 (1H, dd, J=11.1, 4.0 Hz, H-3), 3.00 (1H, td, J=11.0, 4.6 Hz, H-19), 1.69 (3H, s, H-30), 1.01 (3H, s, H-23), 0.99 (3H, s, H-27), 0.95 (3H, s, H-26), 0.85 (3H, s, H-25), 0.81 (3H, s, H-24), 0.73 (1H, d, J=9.5 Hz, H-5). ¹³C NMR: see Table I. HR-ESI-MS m/z 773.4444 [M+Na]⁺ (calcd for C₄₁H₆₆O₁₂Na, 773.4447).

### Synthesis of 28-O-β-D-Glucopyranosyl betulinic acid 3β-O-α-L-rhamnopyranoside (Compound 19)

This compound was prepared from 18b (147 mg, 0.0986 mmol) in the same manner as that described for compound 16a. Purification by C-18 reversed phase flash chromatography (MeOH/H₂O 3:2 to 9:1) gave 19 (61 mg, 81%) as a white amorphous powder. R_{f} 0.78 (CH₂Cl₂/MeOH 3:1); [α]²⁵_{D} −32.4° (c 0.1, MeOH); ¹H NMR (CD₃OD, 400 MHz) δ: 5.49 (1H, d, J=8.1 Hz, H-1″), 4.71 (1H, m, H-1′), 4.71 (1H, m, H-29), 4.59 (1H, m, H-29), 3.84 (1H, m, H-6″), 3.82 (1H, m, H-2′), 3.70 (1H, m, H-5′), 3.70 (1H, m, H-6″), 3.63 (1H, dd, J=9.5, 3.3 Hz, H-3′), 3.43 (1H, m, H-3″), 3.38 (1H, m, H-4″), 3.37 (1H, m, H-5″), 3.36 (1H, m, H-4′), 3.31 (1H, m, H-2″), 3.06 (1H, dd, J=11.6, 4.8 Hz, H-3), 3.00 (1H, td, J=10.8, 6.2 Hz, H-19), 1.69 (3H, s, H-30), 1.22 (3H, d, J=6.2 Hz, H-6′), 1.00 (3H, s, H-27), 0.95 (3H, s, H-26), 0.93 (3H, s, H-23), 0.86 (3H, s, H-25), 0.77 (3H, s, H-24). ¹³C NMR: see Table I. HR-ESI-MS m/z 787.4607 [M+Na]⁺ (calcd for C₄₂H₆₈O₁₂Na, 787.4608).

### Synthesis of 28-O-β-D-Glucopyranosyl betulin 3β-O-β-D-glucopyranoside (Compound 21a)

A solution of the acceptor 1 (250 mg, 0.565 mmol) in anhydrous CH₂Cl₂ (11.3 mL) was stirred for 60 min with 4 Å MS at −10° C. (ice water/acetone bath). TMSOTf (20 μL, 0.113 mmol) was added under an argon atmosphere while keeping rigorous anhydrous conditions. Then, a solution of the donor 4 (1.26 g, 1.70 mmol) in anhydrous CH₂Cl₂ (8.5 mL) was added dropwise over 5 min with continuous stirring. The reaction was allowed to warm to room temperature over 4 h, quenched by addition of Et₃N (0.31 mL, 2.3 mmol) and the solvents were evaporated under reduced pressure. The resulting residue was immediately dissolved in a solution of MeOH/THF/H₂O 1:2:1 (37 mL) to which was added NaOH (438 mg, 11.0 mmol). The reaction mixture was stirred overnight at room temperature and then acidified to pH=4 with aqueous HCl 10%. The solvents were evaporated under reduced pressure to give a solid residue which was purified by C-18 reversed phase flash chromatography (MeOH/H₂O 7:3 to 9:1) to afford 21a (363 mg, 84%, 2 steps) as a white amorphous powder. R_{f} 0.67 (CH₂Cl₂/MeOH 3:1); [α]²⁵_{D} 1.2° (c 0.5, MeOH); ¹H NMR (Pyr-d₅, 400 MHz) δ: 5.05 (1H, d, J=7.6 Hz, H-1″), 4.98 (1H, d, J=7.8 Hz, H-1′), 4.83 (1H, d, J=2.1 Hz, H-29), 4.71 (1H, br s, H-29), 4.67 (1H, m, H-6″), 4.63 (1H, m, H-6′), 4.49 (1H, dd, J=12.1, 5.1 Hz, H-6″), 4.45 (1H, dd, J=11.6, 5.3 Hz, H-6′), 4.34 (1H, m, H-3″), 4.34 (1H, m, H-4″), 4.28 (1H, m, H-3′), 4.27 (1H, m, H-4′), 4.14 (1H, m, H-2″), 4.13 (1H, m, H-5″), 4.10 (1H, m, H-28), 4.08 (1H, m, H-2′), 4.03 (1H, m, H-5′), 3.95 (1H, d, J=9.7 Hz, H-28), 3.43 (1H, dd, J=11.4, 4.3 Hz, H-3), 1.72 (3H, s, H-30), 1.33 (3H, s, H-23), 1.03 (3H, s, H-27), 1.01 (3H, s, H-24), 0.94 (3H, s, H-26), 0.80 (3H, s, H-25), 0.74 (1H, br d, J=8.9 Hz, H-5). ¹³C NMR: see Table I. HR-ESI-MS m/z 789.4747 [M+Na]⁺ (calcd for C₄₂H₇₀O₁₂Na, 789.4758).

### Synthesis of 28-O-β-D-Glucopyranosyl betulinic acid 3β-O-β-D-glucopyranoside (Compound 21b)

This compound was prepared from the acceptor 2 (50 mg, 0.109 mmol) and the donor 4 (243 mg, 0.328 mmol) in the same manner as that described for compound 21a. Purification by C-18 reversed phase flash chromatography (MeOH/H₂O 7:3 to 17:3) gave 21b (49 mg, 58%, 2 steps) as a white amorphous powder. R_{f} 0.66 (CH₂Cl₂/MeOH 3:1); [α]²⁵_{D} −6.8° (c 0.1, MeOH); ¹H NMR (CD₃OD, 400 MHz) δ: 5.49 (1H, d, J=8.1 Hz, H-1″), 4.71 (1H, br s, H-29), 4.60 (1H, br s, H-29), 4.30 (1H, d, J=7.6 Hz, H-1′), 3.84 (1H, m, H-6″), 3.83 (1H, m, H-6′), 3.70 (1H, dd, J=11.9, 3.0 Hz, H-6″), 3.65 (1H, dd, J=11.9, 5.3 Hz, H-6′), 3.42 (1H, m, H-3″), 3.39 (1H, m, H-4″), 3.38 (1H, m, H-5″), 3.33 (1H, m, H-3′), 3.31 (1H, m, H-2″), 3.28 (1H, m, H-4′), 3.24 (1H, m, H-5′), 3.18 (1H, m, H-2′), 3.15 (1H, m, H-3), 3.01 (1H, td, J=10.8, 4.5 Hz, H-19), 1.69 (3H, s, H-30), 1.03 (3H, s, H-23), 0.99 (3H, s, H-27), 0.95 (3H, s, H-26), 0.86 (3H, s, H-25), 0.82 (3H, s, H-24). ¹³C NMR: see Table I. HR-ESI-MS m/z 803.4537 [M+Na]⁺ (calcd for C₄₂H₆₈O₁₃Na, 803.4552).

### Synthesis of 28-O-α-L-Rhamnopyranosyl betulin 3β-O-α-L-rhamnopyranoside (Compound 22a)

This compound was prepared from the acceptor 1 (100 mg, 0.226 mmol) and the donor 8 (421 mg, 0.678 mmol) in the same manner as that described for compound 21a. Purification by C-18 reversed phase flash chromatography (MeOH/H₂O 3:2 to 9:1) gave 22a (53 mg, 32%, 2 steps) as a white amorphous powder. R_{f} 0.87 (CH₂Cl₂/MeOH 3:1); [α]²⁵_{D} −58.4° (c 0.1, CHCl₃/MeOH 1:1); ¹H NMR (CDCl₃/CD₃OD 1:1, 400 MHz) δ: 4.76 (1H, br s, H-1′), 4.70 (1H, m, H-1″), 4.70 (1H, m, H-29), 4.59 (1H, m, H-29), 3.88 (1H, m, H-2′), 3.88 (1H, m, H-2″), 3.75 (1H, dd, J=9.4, 6.2 Hz, H-5″), 3.69 (1H, m, H-3′), 3.69 (1H, m, H-3″), 3.60 (1H, dd, J=9.4, 6.4 Hz, H-5′), 3.51 (1H, d, J=9.2 Hz, H-28), 3.43 (1H, m, H-28), 3.40 (1H, m, H-4′), 3.38 (1H, m, H-4″), 3.07 (1H, dd, J=11.6, 4.8 Hz, H-3), 2.47 (1H, m, H-19), 1.69 (3H, s, H-30), 1.33 (3H, d, J=6.2 Hz, H-6′), 1.26 (3H, d, J=6.4 Hz, H-6″), 1.03 (3H, s, H-26), 0.99 (3H, s, H-27), 0.92 (3H, s, H-23), 0.84 (3H, s, H-25), 0.76 (3H, s, H-24), 0.72 (1H, br d, J=10.0 Hz, H-5). ¹³C NMR (CDCl₃/CD₃OD 1:1, 100 MHz) 5: see Table I. HR-ESI-MS m/z 757.4843 [M+Na]⁺ (calcd for C₄₂H₇₀O₁₀Na, 757.4861).

### Synthesis of 28-O-α-L-Rhamnopyranosyl betulinic acid 3β-O-α-L-rhamnopyranoside (Compound 22b)

This compound was prepared from the acceptor 2 (100 mg, 0.219 mmol) and the donor 8 (408 mg, 0.657 mmol) in the same manner as that described for compound 21a. Purification by C-18 reversed phase flash chromatography (MeOH 7:3 to 17:3) gave 22b (60 mg, 37%, 2 steps) as a white amorphous powder. R_{f} 0.90 (CH₂Cl₂/MeOH 3:1); [α]²⁵_{D} −47.0° (c 0.5, MeOH); ¹H NMR (CD₃OD, 400 MHz) δ: 6.00 (1H, d, J=1.6 Hz, H-1″), 4.75 (1H, d, J=1.3 Hz, H-29), 4.72 (1H, d, J=1.3 Hz, H-1′), 4.62 (1H, br s, H-29), 3.82 (1H, dd, J=3.2, 1.6 Hz, H-2′), 3.79 (1H, dd, J=3.3, 1.9 Hz, H-2″), 3.70 (1H, m, H-5′), 3.67 (1H, m, H-3″), 3.67 (1H, m, H-5″), 3.63 (1H, m, H-3′), 3.46 (1H, t, J=9.4 Hz, H-4″), 3.36 (1H, t, J=9.4 Hz, H-4′), 3.07 (1H, dd, J=11.6, 4.9 Hz, H-3), 3.02 (1H, td, J=10.7, 4.6 Hz, H-19), 1.71 (3H, s, H-30), 1.27 (3H, d, J=6.2 Hz, H-6″), 1.22 (3H, d, J=6.2 Hz, H-6′), 1.02 (3H, s, H-27), 0.94 (3H, s, H-26), 0.93 (3H, s, H-23), 0.87 (3H, s, H-25), 0.77 (3H, s, H-24). ¹³C NMR: see Table I. HR-ESI-MS m/z 771.4639 [M+Na]⁺ (calcd for C₄₂H₆₈O₁₁Na, 771.4654).

TABLE I
¹³C NMR data of bidesmosidic saponins 3, 16a, 16b, 19, 21a, 21b, 22a and 22b^{a}

| Position | 3^{b} | | 16a^{b} | | 16b^{b} | | 19^{c} | | 21a^{d} | | 21b^{c} | | 22a^{a} | | 22b^{c} | |
|---|---|---|---|---|---|---|---|---|---|---|---|---|---|---|---|---|
| 1 | 39.5 | (t) | 39.2 | (t) | 39.1 | (t) | 39.9 | (t) | 39.4 | (t) | 40.1 | (t) | 39.0 | (t) | 39.9 | (t) |
| 2 | 26.7 | (t) | 26.4 | (t) | 26.0 | (t) | 26.8 | (t) | 27.1 | (t) | 27.2 | (t) | 25.9 | (t) | 26.8 | (t) |
| 3 | 90.3 | (d) | 90.2 | (d) | 89.7 | (d) | 90.4 | (d) | 89.2 | (d) | 90.9 | (d) | 89.7 | (d) | 90.4 | (d) |
| 4 | 39.8 | (s) | 39.6 | (s) | 39.5 | (s) | 40.2 | (s) | 40.0 | (s) | 40.3 | (s) | 39.4 | (s) | 40.2 | (s) |
| 5 | 56.5 | (d) | 56.1 | (d) | 55.9 | (d) | 56.9 | (d) | 56.2 | (d) | 57.2 | (d) | 55.8 | (d) | 56.9 | (d) |
| 6 | 18.8 | (t) | 18.6 | (t) | 18.7 | (t) | 19.4 | (t) | 18.8 | (t) | 19.3 | (t) | 18.6 | (t) | 19.4 | (t) |
| 7 | 35.0 | (t) | 34.6 | (t) | 34.6 | (t) | 35.5 | (t) | 34.8 | (t) | 35.5 | (t) | 34.6 | (t) | 35.6 | (t) |
| 8 | 41.5 | (s) | 41.4 | (s) | 41.4 | (s) | 42.0 | (s) | 41.5 | (s) | 42.1 | (s) | 41.3 | (s) | 42.0 | (s) |
| 9 | 51.4 | (d) | 50.8 | (d) | 50.9 | (d) | 52.0 | (d) | 51.0 | (d) | 52.0 | (d) | 50.8 | (d) | 51.9 | (d) |
| 10 | 37.6 | (s) | 37.3 | (s) | 37.3 | (s) | 38.1 | (s) | 37.4 | (s) | 38.1 | (s) | 37.2 | (s) | 38.1 | (s) |
| 11 | 21.6 | (t) | 21.3 | (t) | 21.3 | (t) | 22.1 | (t) | 21.3 | (t) | 22.1 | (t) | 21.2 | (t) | 22.2 | (t) |
| 12 | 26.3 | (t) | 25.7 | (t) | 25.7 | (t) | 26.9 | (t) | 26.0 | (t) | 26.9 | (t) | 25.6 | (t) | 26.9 | (t) |
| 13 | 38.8 | (d) | 38.0 | (d) | 38.0 | (d) | 39.4 | (d) | 38.0 | (d) | 39.4 | (d) | 38.0 | (d) | 40.0 | (d) |
| 14 | 43.1 | (s) | 43.1 | (s) | 43.1 | (s) | 43.6 | (s) | 43.3 | (s) | 43.6 | (s) | 43.1 | (s) | 43.7 | (s) |
| 15 | 30.2 | (t) | 27.5 | (t) | 27.5 | (t) | 30.8 | (t) | 28.0 | (t) | 30.9 | (t) | 27.5 | (t) | 30.8 | (t) |
| 16 | 32.4 | (t) | 29.9 | (t) | 29.9 | (t) | 32.8 | (t) | 30.4 | (t) | 32.8 | (t) | 30.1 | (t) | 33.1 | (t) |
| 17 | 57.4 | (s) | 47.6 | (s) | 47.6 | (s) | 57.9 | (s) | 48.1 | (s) | 58.0 | (s) | 47.3 | (s) | 58.3 | (s) |
| 18 | 50.1 | (d) | 49.3 | (d) | 49.3 | (d) | 50.6 | (d) | 49.5 | (d) | 50.6 | (d) | 49.2 | (d) | 50.5 | (d) |
| 19 | 47.7 | (d) | 48.3 | (d) | 48.3 | (d) | 48.4 | (d) | 48.4 | (d) | 48.4 | (d) | 48.3 | (d) | 48.8 | (d) |
| 20 | 151.1 | (s) | 151.0 | (s) | 151.0 | (s) | 151.8 | (s) | 151.3 | (s) | 151.9 | (s) | 150.8 | (s) | 151.5 | (s) |
| 21 | 31.0 | (t) | 30.1 | (t) | 30.1 | (t) | 31.5 | (t) | 30.5 | (t) | 31.5 | (t) | 30.3 | (t) | 31.8 | (t) |
| 22 | 37.1 | (t) | 35.1 | (t) | 35.1 | (t) | 37.5 | (t) | 35.6 | (t) | 37.5 | (t) | 35.3 | (t) | 38.0 | (t) |
| 23 | 28.2 | (q) | 28.2 | (q) | 28.3 | (q) | 28.7 | (q) | 28.5 | (q) | 28.4 | (q) | 28.3 | (q) | 28.7 | (q) |
| 24 | 16.5 | (q) | 16.5 | (q) | 16.4 | (q) | 16.8 | (q) | 16.4 | (q) | 16.8 | (q) | 16.4 | (q) | 16.8 | (q) |
| 25 | 16.6 | (q) | 16.5 | (q) | 16.4 | (q) | 16.8 | (q) | 17.2 | (q) | 16.8 | (q) | 16.4 | (q) | 16.8 | (q) |
| 26 | 16.3 | (q) | 16.4 | (q) | 16.3 | (q) | 16.7 | (q) | 16.7 | (q) | 16.7 | (q) | 16.2 | (q) | 16.8 | (q) |
| 27 | 15.1 | (q) | 15.1 | (q) | 15.1 | (q) | 15.2 | (q) | 15.3 | (q) | 15.2 | (q) | 15.0 | (q) | 15.2 | (q) |
| 28 | 175.9 | (s) | 68.9 | (t) | 68.8 | (t) | 176.1 | (s) | 68.9 | (t) | 176.2 | (s) | 66.4 | (t) | 175.6 | (s) |
| 29 | 110.1 | (t) | 110.0 | (t) | 109.9 | (t) | 110.3 | (t) | 110.4 | (t) | 110.3 | (t) | 110.0 | (t) | 110.6 | (t) |
| 30 | 19.5 | (q) | 19.4 | (q) | 19.3 | (q) | 19.5 | (q) | 19.6 | (q) | 19.5 | (q) | 19.3 | (q) | 19.6 | (q) |
| 1′ | 106.2 | (d) | 105.5 | (d) | 103.3 | (d) | 104.4 | (d) | 107.3 | (d) | 106.8 | (d) | 103.1 | (d) | 104.4 | (d) |
| 2′ | 72.1 | (d) | 71.7 | (d) | 71.5 | (d) | 72.5 | (d) | 76.2 | (d) | 75.7 | (d) | 71.4 | (d) | 72.5 | (d) |
| 3′ | 73.6 | (d) | 73.1 | (d) | 71.9 | (d) | 72.5 | (d) | 79.2 | (d) | 78.3 | (d) | 71.9 | (d) | 72.6 | (d) |
| 4′ | 68.4 | (d) | 67.8 | (d) | 73.4 | (d) | 74.1 | (d) | 72.2 | (d) | 71.7 | (d) | 73.3 | (d) | 74.1 | (d) |
| 5′ | 65.4 | (t) | 64.9 | (t) | 68.8 | (d) | 69.9 | (d) | 78.7 | (d) | 77.7 | (d) | 68.8 | (d) | 69.9 | (d) |
| 6′ | — | | — | | 17.5 | (q) | 17.9 | (q) | 63.4 | (t) | 62.8 | (t) | 17.7 | (q) | 17.9 | (q) |
| 1″ | 94.6 | (d) | 104.3 | (d) | 104.4 | (d) | 95.2 | (d) | 106.4 | (d) | 95.2 | (d) | 101.1 | (d) | 95.1 | (d) |
| 2″ | 73.4 | (d) | 74.2 | (d) | 74.2 | (d) | 74.1 | (d) | 75.8 | (d) | 74.1 | (d) | 71.3 | (d) | 71.4 | (d) |
| 3″ | 77.7 | (d) | 76.9 | (d) | 77.0 | (d) | 78.4 | (d) | 79.0 | (d) | 78.4 | (d) | 71.8 | (d) | 72.8 | (d) |
| 4″ | 70.6 | (d) | 70.8 | (d) | 70.8 | (d) | 71.1 | (d) | 72.2 | (d) | 71.1 | (d) | 73.1 | (d) | 73.4 | (d) |
| 5″ | 78.0 | (d) | 76.3 | (d) | 76.5 | (d) | 78.8 | (d) | 79.0 | (d) | 78.8 | (d) | 68.7 | (d) | 69.9 | (d) |
| 6″ | 62.0 | (t) | 62.3 | (t) | 62.1 | (t) | 62.4 | (t) | 63.3 | (t) | 62.4 | (t) | 17.5 | (q) | 18.2 | (q) |

^{a}Spectra recorded at 100 MHz. The multiplicities were deduced from DEPT experiments.
^{b}CDCl₃/CD₃OD
^{c}CD₃OD
^{d}Pyridine-d₅

### Cell Lines and Culture Conditions

Human lung carcinoma (A549, ATCC # CCL-185™), human colorectal adenocarcinoma (DLD-1, ATCC # CCL-221™), human breast adenocarcinoma (MCF7, ATCC # HTB-22™), human prostate adenocarcinoma (PC-3, ATCC # CCL-1435™) and human normal skin fibroblasts (WS1, ATCC # CRL-1502™) cell lines were obtained from the American Type Culture Collection (ATCC). All cell lines were cultured in minimum essential medium containing Earle's salts and L-glutamine (Mediatech Cellgro, VA), to which were added 10% fetal bovine serum (Hyclone), vitamins (1×), penicillin (100 IU/mL) and streptomycin (100 μg/mL), essential amino acids (1×), and sodium pyruvate (1×) (Mediatech Cellgro, VA). Cells were kept at 37° C. in a humidified environment containing 5% CO₂.

### Cytotoxicity Assay

Exponentially growing cells were plated in 96-well microplates (Costar, Corning Inc.) at a density of 5×10³ cells per well in 100 μL of culture medium and were allowed to adhere for 16 h before treatment. Increasing concentrations of each compound in biotechnology performed certified dimethyl sulfoxide (DMSO) (Sigma-Aldrich, Cat. # D2438) and the cells were incubated for 48 h. The final concentration of DMSO in the culture medium was maintained at 0.5% (v/v) to avoid solvent toxicity. Cytotoxicity was assessed using resazurin (O'Brien, J. et al., 2000. Eur J Biochem 267(17): 5421-6) on an automated 96-well Fluoroskan Ascent F1™ plate reader (Labsystems) using excitation and emission wavelengths of 530 and 590 nm, respectively. Fluorescence was proportional to the cellular metabolic activity in each well. Survival percentage was defined as the fluorescence in experimental wells compared to that in control wells after subtraction of blank values. Each experiment was carried out twice in triplicate. IC₅₀ results are expressed as mean±standard deviation.

### Example 2

### Synthesis of Bidesmosides

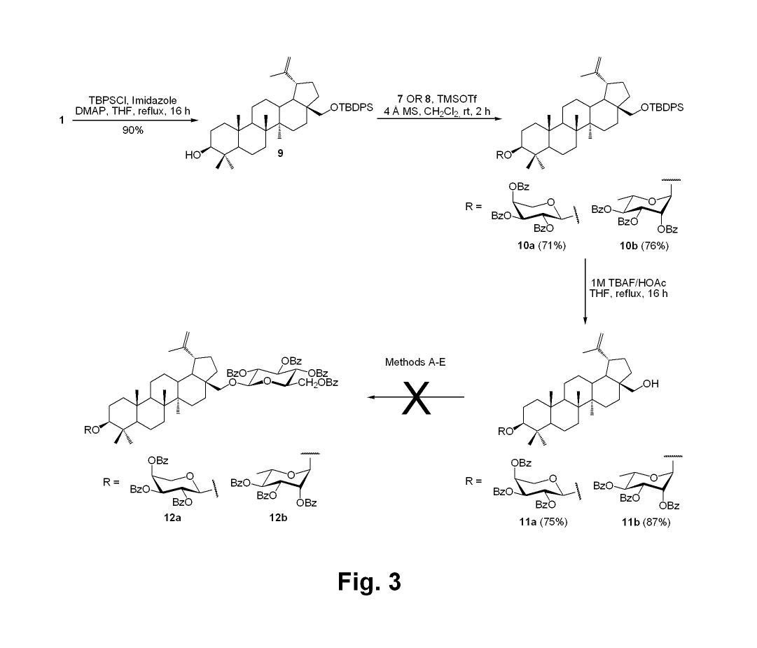

In order to synthesize bidesmosidic betulin saponins, it was first tried to introduce arabinopyranosyl or rhamnopyranosyl moieties at the C-3 position of 1 prior to glucosylating the C-28 position. As revealed in FIG. 3, betulin (1) (Gauthier et al., 2006, supra) was treated with tert-butyldiphenylsilyl chloride (TBDPSCl) in conjunction with imidazole and 4-dimethylaminopyridine (DMAP) in refluxing tetrahydrofuran (THF) to give 9 (90%) protected at the C-28 primary hydroxyl position (Zhang, Y. et al., Carbohydr. Res. 2004, 339: 1753-1759). The latter was glycosylated with the known 2,3,4-tri-O-benzoyl-β-L-arabinopyranosyl trichloroacetimidate (7) (Yu, B. et al., J. Am. Chem. Soc. 1999, 121: 12196-12197) or 2,3,4-tri-O-α-L-rhamnopyranosyl trichloroacetimidate (8) (Ziegler, T. et al., Tetrahedron: Asymmetry 1998, 9: 765-780) under the promotion of the Lewis acid trimethylsilyl trifluoromethanesulfonate (TMSOTf) in dry dichloromethane (CH₂Cl₂) at room temperature to afford protected monodesmosides 10a and 10b in good yields (71% and 76%, respectively). Desilylation of 10a and 10b under standard conditions (Zhang, Y. et al., 2004, supra), i.e. tetrabutylammonium bromide (TBAF) and acetic acid (HOAc) in refluxing THF, readily furnished benzoylated betulin saponins 11a (75%) and 11b (87%). Since the next step consisted in the glucosylation at the C-28 position, it was attempted to couple the known donor 2,3,4,6-tetra-O-benzoyl-α-D-glucopyranosyl trichloroacetimidate (4) (Fukase, K. et al., Chem. Express 1993, 8: 409-412) with acceptor 11a using the above-mentioned glycosylation conditions. However, the reaction afforded the rearrangement product allobetulin 3β-O-2,3,4-tri-O-benzoyl-α-L-arabinopyranoside in 42% yield without any trace of the desired bidesmosidic glycoside 12a. Similar treatment of acceptor 11b with donor 4 led to the exclusive formation of the trans-esterification product 28-O-benzoyl betulin 3β-O-2,3,4-tri-O-benzoyl-α-L-rhamnopyranoside in 42% yield. As shown in FIG. 3, further modifications of the glycosylation conditions were considered using acceptor 11b in conjunction with various glucosyl donors (4-6) (FIG. 2) and promoters such as boron trifluoride diethyl etherate (BF₃.OEt₂) and silver trifluoromethanesulfonate (AgOTf). Also, both Schmidt's inverse procedure (Schmidt, R. R. and Toepfer, A. Tetrahedron Lett. 1991, 32: 3353-3356) and phase-transfer conditions (Bliard, C. et al., Tetrahedron Lett. 1994, 35: 6107-6108) were tried in order to glucosylate the C-28 position of 11b. Unfortunately, all these attempts failed to yield the target bidesmoside 12b. Instead, the rapid decomposition of sugar donors 4-6 was generally observed based on TLC analysis. It is worth noting that 11b was nearly quantitatively transformed into allobetulin 3β-O-2,3,4-tri-O-benzoyl-α-L-rhamnopyranoside when the Lewis acid AgOTf was used as promoter of the glycosylation reaction. Interestingly, the yields of the rearrangement are comparable to those reported by Li and co-workers for the preparation of allobetulin from betulin (1) catalysed by solid acids (Li, T.-S. et al., J. Chem. Soc., Perkin Trans 1998, 1: 3957-3965).

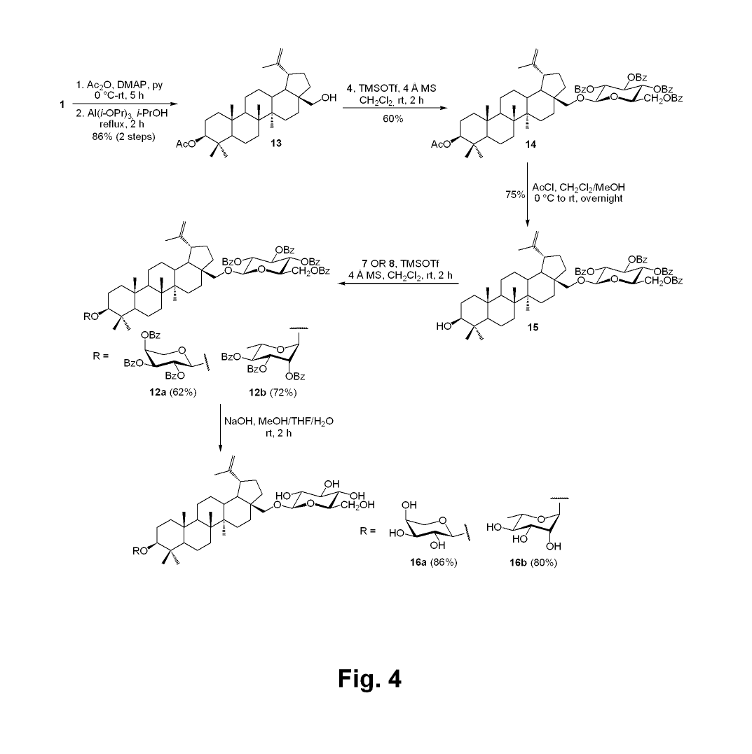

Therefore, another approach was tried for the synthesis of bidesmosidic betulin saponins. According to FIG. 4, the known betulin 3-acetate (13) (Thibeault D. et al., 2007, supra) was prepared in good yield (86%, two steps) from betulin (1) following a reported procedure. Once again, attempts to glucosylate acceptor 13 with donor 4 under the catalytic action of TMSOTf (0.1 equiv) in dry CH₂Cl₂ (20 mL/mmol) afforded rearrangement (allobetulin 3-acetate, 30% yield) and trans-esterification (28-O-benzoyl betulin 3-acetate, 17% yield) products instead of the desired glycoside 14. However, it was found that condensation of 13 and 4 proceeded smoothly to furnish 14 in a convenient 60% yield when only 0.05 equiv of TMSOTf was used in 40 mL/mmol of dry CH₂Cl₂. Thereafter, deacetylation of the C-3 position was achieved by treatment of 14 with acetyl chloride (AcCl) (Du, Y. et al., J. Org. Chem. 2004, 69: 2206-2209) in a dry solution of CH₂Cl₂/MeOH 1:2 to afford 15 in good yield (75%). The latter acceptor was coupled with donors 7 or 8 using TMSOTf as the promoter to give the fully benzoylated bidesmosides 12a (62%) and 12b (72%) which were deprotected using standard conditions (NaOH, MeOH/THF/H₂O 1:2:1) to provide the target bidesmosidic betulin saponins 16a and 16b in good yields (86% and 80%, respectively). The overall yields for the syntheses were 24% for 16a and 26% for 16b over four linear steps starting from betulin 3-acetate (13).

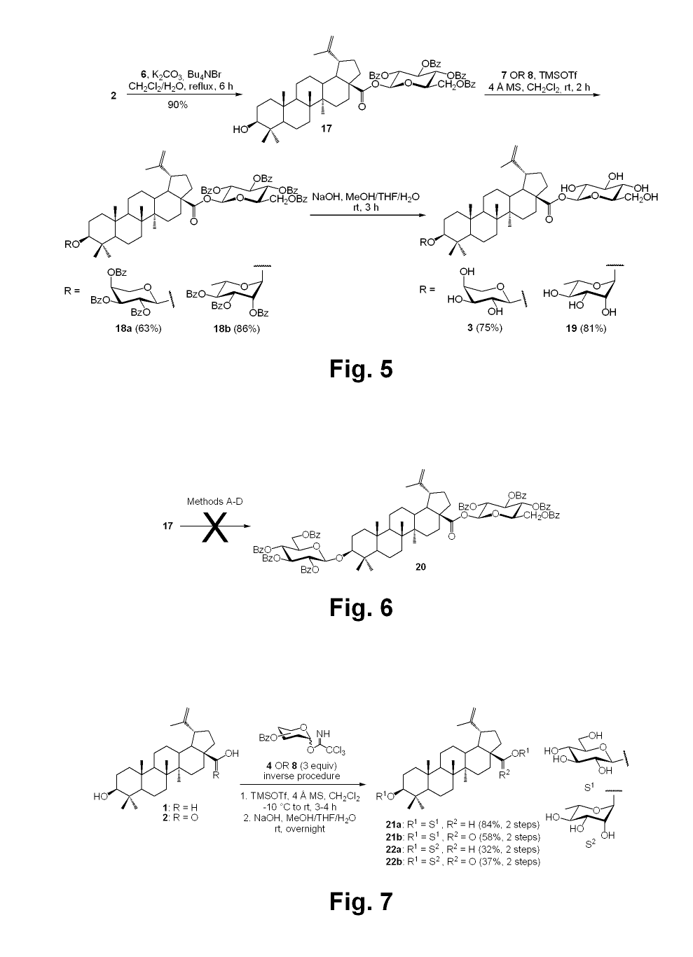

The synthesis of the natural bidesmosidic betulinic acid saponin 3 along with the non-natural saponin 19 was performed as follows. As depicted in FIG. 5, the lupane-type triterpenoid betulinic acid (2) was condensed with the known donor 2,3,4,6-tetra-O-benzoyl-α-D-glucopyranosyl bromide (6) (Fletcher, H. G. Meth. Carbohydr. Chem. 1963, 2: 226-228) under phase-transfer conditions (Bliard, C. et al., Tetrahedron Lett. 1994, 35: 6107-6108) using potassium carbonate (K₂CO₃) and TBAF in a refluxing solution of CH₂Cl₂/H₂O 1:1 to furnish 17 in a yield of 90%. The latter was coupled with donors 7 or 8 under the promotion of TMSOTf to afford 18a (63%) and 18b (86%). Subsequent deprotection of the benzoylated groups by treatment with NaOH in MeOH/THF/H₂O provided the target bidesmosidic betulinic saponins 3 (75%) and 19 (81%). The overall yields for the syntheses were 43% for 3 and 63% for 19 over three linear steps starting from betulinic acid (1). Unexpectedly, it was found that the physical and analytical data (¹H NMR, ¹³C NMR and [α]²⁵_{D}) of saponin 3 were not in agreement with those reported for the natural product isolated from Schefflera rotundifolia (Braca, A. et al., Planta Med. 2004, 70: 960-966).

Surprisingly, the glucosylation at the C-3 position of 28-O-2,3,4,6-tetra-O-benzoyl-β-D-glucopyranosyl betulinic acid (17) was proved to be difficult. In fact, as shown in FIG. 6, attempts to condensate acceptor 17 with either the trichloroacetimidate sugar donor 4 under Schmidt's normal (Schmidt, R. R. Adv. Carbohydr. Chem. Biochem. 1994, 50: 21-123) and inverse procedure (Schmidt, R. R. and Toepfer, A. Tetrahedron Lett. 1991, 32: 3353-3356) or the bromide sugar donor 6 in conjunction with silver oxide (Ag₂O) (Wang, P. et al., J. Org. Chem. 2005, 70: 8884-8889) and AgOTf (modified Koenigs-Knorr methods, Li, C. et al., Carbohydr. Res. 1998, 306: 189-195) failed to yield the fully protected bidesmosidic betulinic acid saponin 20. According to TLC and NMR analysis, no coupling product was observed in all assays while acceptor 17 was nearly fully recovered. Thus, another strategy was adopted in which the unprotected betulin (1) and betulinic acid (2) are glycosylated at both C-3 and C-28 positions via Schmidt's inverse procedure (Schmidt, R. R. et al., 1991, supra) (FIG. 7). Using this methodology, the acceptors (1 or 2) and the promoter (TMSOTf) were premixed before the dropwise addition of the sugar donors (4 or 8, 3 equiv) at low temperature (−10° C.). Deprotection of the crude resulting product (NaOH, MeOH/THF/H₂O) and purification by C-18 inversed phase flash chromatography afforded the target saponins (21a, 21b, 22a, 22b) in yields ranging from 37% to 84% over two steps. The 1,2-trans-glycosidic linkage (α-L-rhamnoside and β-D-glucoside) of saponins was clearly demonstrated by ¹H NMR analysis (δ 4.98, d, J_{1,2} 7.8 Hz and δ 4.30, d, J_{1,2} 7.6 Hz, H-1′ for 21a and 21b; δ 4.76, br s and δ 4.72, d, J_{1,2} 1.3 Hz, H-1′ for 22a and 22b) (Agrawal, P. K. Phytochemistry 1992, 31: 3307-3330). The purity of newly synthesized saponins used in the cytotoxic assays was found to be >95% acceptable, as measured by HPLC (two methods).

### Example 3

### Cytotoxic Activity of the Bidesmosidic Saponins

In vitro cytotoxic activity of lupane-type bidesmosidic saponins was evaluated against four human cancer cell lines including lung carcinoma (A549), and colorectal (DLD-1), breast (MCF7) and prostate (PC-3) adenocarcinomas. The parent triterpenoids betulin (1) (Gauthier et al., 2006, supra), betulinic acid (2) (Kessler, J. H. et al., Cancer Lett. 2007, 251: 132-145) and the clinically used etoposide were used as positive controls. The cytotoxicity of 28-O-β-D-glucopyranosides of betulin (Gauthier et al., 2006, supra) and betulinic acid (Baglin et al., 2003, supra) was also investigated. The cell viability was assessed through resazurin reduction test (O'Brien et al, 2000, supra) after 48 hours of incubation between the compounds and cells. Since resazurin (Alamar blue) is a nontoxic dye, measurements can be obtained without killing the cells as opposed to the standard MTT assay (Bellamy, W. T. Drugs 1992, 44: 690-708). The cytotoxicity results were expressed as the concentration inhibiting 50% of the cell growth (IC₅₀).

It was previously shown in prior structure-activity relationships (SAR) studies that the free C-28 carboxylic acid function is important to preserve the cytotoxic activity of betulinic acid (2) ((Baglin et al., 2003, supra; Kim, D. S. H. L. et al., Bioorg. Med. Chem. Lett. 1998, 8:1707-1712; Chatterjee, P. et al., J. Nat. Prod. 1999, 62: 761-763; Urban, M. et al., Bioorg. Med. Chem. 2005, 13: 5527-5535). As revealed in Table II, monodesmosidic betulin (1) and betulinic acid (2) saponins bearing a β-D-glucopyranoside moiety at the C-28 position show low or no cytotoxicity (IC₅₀>100 μM). Surprisingly, the cytotoxicity profile of the synthesized bidesmosidic saponins, which lack the carboxylic acid function, was generally similar or higher than that of betulinic acid (2) against the tested cancer cell lines (Table II). For example, bidesmosidic derivatives 21a and 21b were preferentially cytotoxic and significantly more active than betulinic acid (2) against breast adenocarcinoma (MCF7) cancer cell lines, with IC₅₀ ranging from 14.5 to 20 μM. It is noteworthy that the betulin derivative 16a showed more potent anticancer activity against MCF7 and PC-3 cell lines (IC₅₀ 9.5 and 5.3 μM, respectively) as compared to the betulinic acid saponin 3 (IC₅₀ 23-76 μM), which bears the same sugar residues. These results show that the relative cytotoxicity of bidesmosidic betulin and betulinic acid saponins are influenced by the nature of both the aglycone and the sugar moieties at the C-3 and C-28 positions.

Bidesmosides 16b, 19, 22a and 22b strongly inhibit the growth of various cancer cell lines (IC₅₀ 1.7-23 μM). Saponin derivatives 22a and 22b containing an α-L-rhamnopyranoside moiety at both C-3 and C-28 positions were highly cytotoxic against all tested cancer cell lines (IC₅₀ 1.7-1.9 and 6.0-7.2 μM, respectively) and significantly more active than their parent triterpenoids (P<0.05). Notably, bidesmosidic betulin saponin 22a was the most potent of all tested compounds to inhibit the growth of human cancer cell lines and its toxicity was also significantly higher (P<0.05) than betulinic acid 3 β-O-α-L-rhamnopyranoside (IC₅₀ 3.8 um, A549).

TABLE II
Cytotoxicity (IC₅₀) of bidesmosidic saponins against different cancer cell lines.

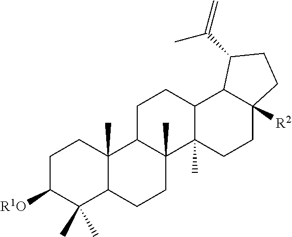

| | | | IC₅₀ (μmol•L⁻¹)^{a} | | | |
|---|---|---|---|---|---|---|
| Compound | R¹ | R² | A549^{b} | DLD-1^{c} | MCF7^{d} | PC-3^{e} |
| 2 | H | 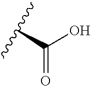 | 10.3 ± 0.4^{g} | 15.0 ± 0.3^{g} | 41 ± 1 | 40 ± 2 |
| 28GBet | H | 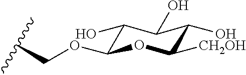 | >100^{g} | >100 | >100 | >100 |
| 28GBetA | H | 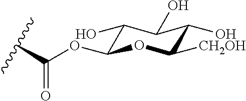 | >100 | >100 | >100 | >100 |
| 21a | 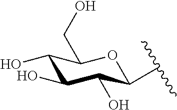 | 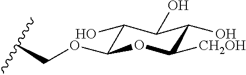 | >100 | 27 ± 2 | 14.5 ± 0.9 | >100 |
| 16b | 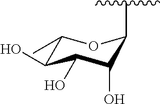 | 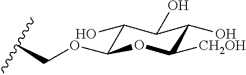 | 16.8 ± 0.9 | 10.6 ± 0.9 | 9.0 ± 0.7 | 6.9 ± 0.4 |
| 16a | 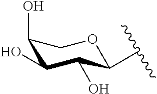 | 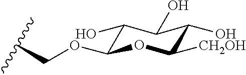 | >100 | 19 ± 2 | 9.5 ± 0.8 | 5.3 ± 0.6 |
| 21b | 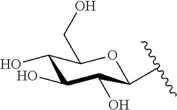 | 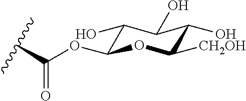 | >100 | >100 | 20 ± 2 | 66 ± 3 |
| 19 | 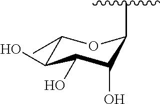 | 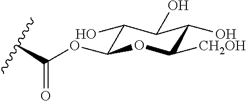 | 23 ± 1 | 11.0 ± 0.5 | 5.7 ± 0.6 | 11.2 ± 0.8 |
| 3 | 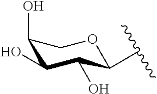 | 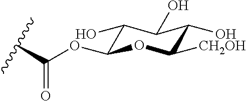 | 76 ± 4 | 60 ± 5 | 23 ± 1 | 68 ± 7 |
| 22a | 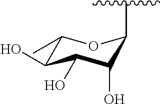 | 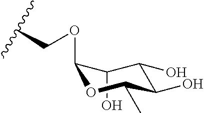 | 1.9 ± 0.1 | 1.9 ± 0.1 | 1.7 ± 0.2 | 1.8 ± 0.1 |
| 22b | 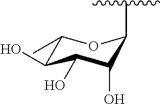 | 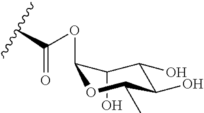 | 7.2 ± 0.5 | 7.3 ± 0.3 | 6.0 ± 0.6 | 7.2 ± 0.5 |

| | | | | |
|---|---|---|---|---|
| Etoposide | 3.4 ± 0.1 | 27 ± 5 | nd^{h} | nd^{h} |

Bet = betulin; BetA = betulinic acid; Glc = β-D-glucopyranose; Rha = α-L-rhamnopyranose; Ara = α-L-arabinopyranose.
^{a}Data represent mean values ± standard deviation for three independent experiments made in triplicate.
^{b}Human lung carcinoma.
^{c}Human colorectal adenocarcinoma.
^{d}Human breast adenocarcinoma.
^{e}Human prostate adenocarcinoma.
^{f}Human skin fibroblasts.
^{g}Results previously reported in Gauthier, C.; Legault, J.; Lebrun, M.; Dufour, P.; Pichette, A. Glycosidation of lupane-type triterpenoids as potent in vitro cytotoxic agents. Bioorganic & Medicinal Chemistry 2006, 14, 6713-6725.
^{h}Not determined.

### Example 4

### Cytotoxicity Against Other Cancer Cell Lines

Compounds presented in Table II are also tested in the following tumour cell lines: U-251 (Human glioma), B-16-F1, HEP G2 (Human hepatocellular carcinoma), PA-1 (Human ovary teratocarcinoma metastatic), MDA-MB-231 (Human breast adenocarcinona metastatic), SK-MEL-2 (Human malignant melanoma); Panc 05.04 (Human pancreas adenocarcinoma), K-562 (Human chronic myelogenous leukaemia), A375.S2 (Human skin malignant melanoma), Caco-2 (Human colorectal adenocarcinoma), U-87 (Human colorectal adenocarcinoma) and IMR-90 (Human lung fibroblast).

### Example 5

### In Vivo Antitumoral Evaluation of Compounds of Table II

Cell lines and mice preparation: The Lewis lung carcinoma cell lines (#CRL-1642, lot # 4372266, ATCC) and the C57BL/6 mouse strain (Charles River Inc., St-Constant, Qc) are used. Cells are grown to 90% confluence in complete DMEM medium containing Earle's salts and L-glutamine (Mediatech Cellgro, VA), 10% foetal bovine serum (Hyclone), vitamins (1×), penicillin (100 I.U./mL) and streptomycin (100 μg/mL), essential amino acids (1×) and sodium pyruvate (1×) (Mediatech Cellgro, VA). Cells are then harvested with up and down only. Cells are counted using a hemacytometer and resuspended in DMEM medium without SVF. 100 μL of a solution containing 1×10⁷ cells/mL are inoculated subcutaneously in the right flank of each 6 weeks old mouse on day zero.

Mice are handled and cared for in accordance with the Guide for the Care and Use of Laboratory Animals. Treatment is performed by IP route starting 1 day after tumour injection. Betulinic acid and compounds of Table II, and in particular compounds 22A and 22B are dissolved in DMSO and administered at 50, 100 and 200 mg/kg of body weight every 3-4 days. Individual dose are based on the body weight of each mouse. All the mice receive a constant injection volume of 100 μL per 25 g of body weight. Control mice are similarly treated IP with the solvent used for the dissolution of drug (DMSO). The experimental mice are weighed daily.

Data analysis: In vivo antitumor activity is evaluated according to the parameters as follows (Miot-Noirault, E. et al. Invest. New Drugs 2004, 22, 369-378):

(a) Calculated tumour weight (CTW): The CTW of each tumour is estimated from two-dimensional measurements performed once a day with a slide calliper, according to the formula: CTW (mg)=(L×W²)/2 with L=length in mm and W=width in mm. Differences in CTW between treated and control groups (DMSO) are analyzed for significance using the U Wilcoxon-Mann-Whitney test and Student t-test. Values of p<0.05 are considered statistically significant.

(b) Treated/Control value (T/C) and Tumour Growth Inhibition (TGI): The T/C is calculated as the ratio of the mean CTW of TW of drug-treated mice versus controls: T/C=(CTW of the drug-treated group on Day X/CTW of the control group on Day X)×100. TGI is 100−(T/C) value.

The toxicity of treatment is determined using the body weight of mice. The National Cancer Institute considers that a treatment is toxic if the loss of weight is superior to 20% with regard to the initial weight.

### Example 6

### Determination of the Maximum Tolerated Dose (MTD) for Compounds of Table II

Groups of five mice (Charles River) receive a single IP injection of compounds of Table II and in particular of compounds 22A and 22B in DMSO at doses of 50, 100, 250 and 500 mg/kg of body weight. Individual dose are based on the body weight of each mouse. A group of five control mice receive the vehicle (DMSO). All the mice receive a constant injection volume of 100 μL per 25 g of body weight. After injection, mice are observed to evaluate general clinical state. For each animal, a score is calculated based on the absence (value 0) or presence (value 1) of diarrhoea, lethargy, rough coat and closed eyes. A clinical state score (CSS) is then calculated per group by summing individual scores. All the mice are weighed daily during 3 days following the injection. The maximal weight loss is determined 24 hours and 3 days following the injection. The MTD is defined as the highest single dose that meets all the following criteria: 1) zero death per group; 2) maximal weight loss 20% in non-tumour bearing animals; and 3) CSS value lower than 15.

The calculated dose can be scaled up to a human equivalent dose (HED) using published conversion tables that take into account the body surface area of the species. The conversion factor from mice to human is 12.3, a MTD of 250 mg/kg for mice for instance is equivalent to 20.33 mg/kg in human. This value (20.33 mg/kg) is divided by a security factor of 10. The calculated MTD would thus be 2.33 mg/kg. For an average human weighting 60 kg, the calculated dose would thus be 139.8 mg.

Although the present invention has been described hereinabove by way of specific embodiments thereof, it can be modified, without departing from the spirit and nature of the subject invention as defined in the appended claims.

1. A compound of formula (I): 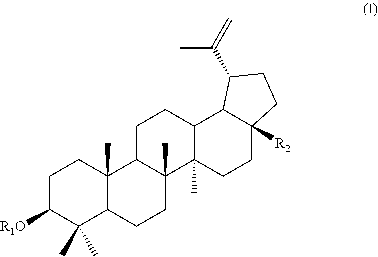
wherein
(i) R₁ is α-L-arabinopyranose and R₂ is CH₂O-β-D-glucopyranose; or
(ii) R₁ is α-L-rhamnopyranose and R₂ is CH₂O-β-D-glucopyranose, COO-β-D-glucopyranose, CH₂O-β-L-rhamnopyranose or COO-6-L-rhamnopyranose,
or a pharmaceutically acceptable salt thereof.

2. The compound of claim 1, wherein R₁ is α-L-arabinopyranose and R₂ is CH₂O-β-D-glucopyranose.

3. The compound of claim 1, wherein R₁ is α-L-rhamnopyranose and R₂ is CH₂O-β-D-Glucopyranose.

4. The compound of claim 1, wherein R₁ is α-L-rhamnopyranose and R₂ is COO-β-D-glucopyranose.

5. The compound of claim 1, wherein R₁ is α-L-rhamnopyranose and R₂ is CH₂O-β-L-rhamnopyranose.

6. The compound of claim 1, wherein R₁ is α-L-rhamnopyranose and R₂ is COO-β-L-rhamnopyranose.

7. A pharmaceutical composition comprising the compound of claim 1 and a pharmaceutically acceptable diluent, carrier or excipient.

8. A method for treating carcinoma comprising administering to a subject in need thereof an effective amount of a compound of formula (I): 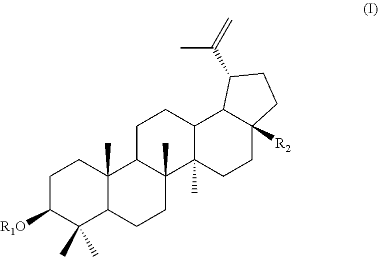
wherein
(i) R₁ is α-L-arabinopyranose and R₂ is CH₂O-β-D-glucopyranose or COO-β-D-glucopyranose; or
(ii) R₁ is α-L-rhamnopyranose and R₂ is CH₂O-β-D-glucopyranose, COO-β-D-glucopyranose, CH₂O-β-L-rhamnopyranose or COO-β-L-rhamnopyranose; or
(iii) R₁ is β-D-glucopyranose and R₂ is CH₂O-β-D-glucopyranose or COO-β-D-glucopyranose,
or a pharmaceutically acceptable salt thereof.

9. The method of claim 8, wherein said carcinoma is lung carcinoma, colorectal adenocarcinoma, breast adenocarcinoma, or prostate adenocarcinoma.

10. The method of claim 9, wherein said carcinoma is breast adenocarcinoma and wherein
(i) R₁ is α-L-arabinopyranose and R₂ is CH₂O-β-D-glucopyranose or COO-β-D-glucopyranose; or
(ii) R₁ is α-L-rhamnopyranose and R₂ is CH₂O-β-D-glucopyranose, COO-β-D-glucopyranose, CH₂O-β-L-rhamnopyranose or COO-β-L-rhamnopyranose; or
(iii) R₁ is β-D-glucopyranose and R₂ is CH₂O-β-D-glucopyranose.

11. The method of claim 9, wherein said carcinoma is lung carcinoma, and wherein
(i) R₁ is α-L-arabinopyranose and R₂ is COO-β-D-glucopyranose; or
(ii) R₁ is α-L-rhamnopyranose and R₂ is CH₂O-β-D-glucopyranose, COO-β-D-glucopyranose, CH₂O-β-L-rhamnopyranose or COO-β-L-rhamnopyranose;

12. The method of claim 9, wherein said carcinoma is prostate adenocarcinoma, and wherein
(i) R₁ is α-L-arabinopyranose and R₂ is CH₂O-β-D-glucopyranose or COO-β-D-glucopyranose; or
(ii) R₁ is α-L-rhamnopyranose and R₂ is CH₂O-β-D-glucopyranose, COO-β-D-glucopyranose, CH₂O-β-L-rhamnopyranose or COO-β-L-rhamnopyranose; or
(iii) R₁ is β-D-glucopyranose and R₂ is COO-β-D-glucopyranose.

13. The method of claim 8, wherein R₁ is α-L-rhamnopyranose and R₂ is CH₂O-β-L-rhamnopyranose.

14. The method of claim 8, wherein the administration is parenteral or systemic.

15. The method of claim 8, wherein the administration is at a tumour site.

16. The method of claim 14, wherein the administration is in a dosage of about 0.5 mg/kg to about 50 mg/kg.

17. The method of claim 16, wherein the administration is in a dosage of about 4 mg/kg to about 40 mg/kg.

18.-34. (canceled)

35. A method of identifying a tumor amenable to treatment with the compound of claim 1, comprising (i) contacting a sample of cells derived from said tumor with the compound, and (ii) determining the IC₅₀ value of the compound against the cells, wherein an IC₅₀ value of about 50 μM or less is indicative that the tumor is amenable to treatment with said compound.

36. The method of claim 35, wherein the IC₅₀ value is 20 μM or less.

37. The method of claim 36, wherein the IC₅₀ value is 10 μM or less.

38. The method of claim 35, wherein said sample of cells is derived from a biopsy sample from a subject.

39. The method of claim 35, wherein said sample of cells is derived from a biological fluid obtained from a subject.

40. A method of inhibiting the growth of a carcinoma cell comprising contacting said cell with an effective amount of a compound of formula (I): 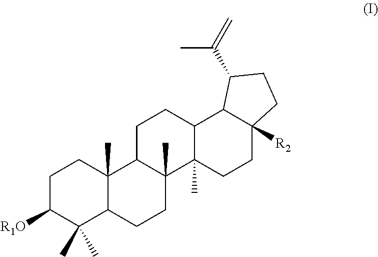
wherein
(i) R₁ is α-L-arabinopyranose and R₂ is CH₂O-β-D-glucopyranose or COO-β-D-glucopyranose; or
(ii) R₁ is α-L-rhamnopyranose and R₂ is CH₂O-β-D-glucopyranose, COO-β-D-glucopyranose, CH₂O-β-L-rhamnopyranose or COO-β-L-rhamnopyranose; or
(iii) R₁ is β-D-glucopyranose and R₂ is CH₂O-β-D-glucopyranose or COO-β-D-glucopyranose,
or a pharmaceutically acceptable salt thereof.

41. The method of claim 40, wherein said carcinoma cell is a lung carcinoma cell, a colorectal adenocarcinoma cell, a breast adenocarcinoma cell, or a prostate adenocarcinoma cell.

42. The method of claim 41, wherein said carcinoma cell is a breast adenocarcinoma cell and wherein
(i) R₁ is α-L-arabinopyranose and R₂ is CH₂O-β-D-glucopyranose or COO-β-D-glucopyranose; or
(ii) R₁ is α-L-rhamnopyranose and R₂ is CH₂O-β-D-glucopyranose, COO-β-D-glucopyranose, CH₂O-β-L-rhamnopyranose or COO-β-L-rhamnopyranose; or
(iii) R₁ is β-D-glucopyranose and R₂ is CH₂O-β-D-glucopyranose.

43. The method of claim 41, wherein said carcinoma cell is a lung carcinoma cell, and wherein
(i) R₁ is α-L-arabinopyranose and R₂ is COO-β-D-glucopyranose; or
(ii) R₁ is α-L-rhamnopyranose and R₂ is CH₂O-β-D-glucopyranose, COO-β-D-glucopyranose, CH₂O-β-L-rhamnopyranose or COO-β-L-rhamnopyranose; or
(iii) R₁ is β-D-glucopyranose and R₂ is CH₂O-β-D-glucopyranose or COO-β-D-glucopyranose.

44. The method of claim 41, wherein said carcinoma cell is a lung carcinoma cell or a prostate adenocarcinoma cell, and wherein
(i) R₁ is α-L-arabinopyranose and R₂ is CH₂O-β-D-glucopyranose or COO-β-D-glucopyranose; or
(ii) R₁ is α-L-rhamnopyranose and R₂ is CH₂O-β-D-glucopyranose, COO-β-D-glucopyranose, CH₂O-β-L-rhamnopyranose or COO-β-L-rhamnopyranose; or
(iii) R₁ is β-D-glucopyranose and R₂ is COO-β-D-glucopyranose.

45. The method of claim 40, wherein R₁ is α-L-rhamnopyranose and R₂ is CH₂O-β-L-rhamnopyranose.

46. The method of claim 40, wherein said compound is present in a pharmaceutical composition.

47. A method for preparing a compound of formula (I): 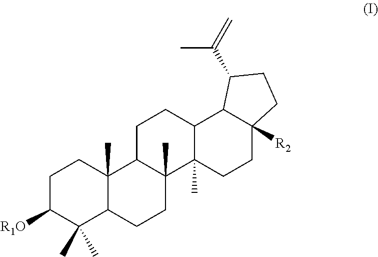
wherein
R₁ is α-L-arabinopyranose or α-L-rhamnopyranose, and
R₂ is CH₂O-β-D-glucopyranose;
said method comprising:
(1)
(a) glycosylating the C-28 position of betulin 3-acetate with a perbenzoylated or peracetylated trichloroacetimidate or trifluorophenylacetimidate glucose donor under the promotion of a Lewis acid to yield a first glycosylated compound;
(b) submitting the first glycosylated compound to regioselective deacetylation conditions to cleave the acetyl group at the C-3 position to yield a deacetylated compound;
(c) glycosylating the C-3 position of the deacetylated compound with a perbenzoylated or peracetylated trichloroacetimidate or trifluorophenylacetimidate arabinose or rhamnose donor under the promotion of a Lewis acid to yield a second glycosylated compound; and
(d) submitting the second glycosylated compound to deacetylation conditions; or
(2)
(a) glycosylating the C-28 position of betulinic acid with a perbenzoylated or peracetylated bromide glucose donor under phase-transfer conditions to yield a first glycosylated compound;
(b) glycosylating the C-3 position of the first glycosylated compound with a perbenzoylated or peracetylated trichloroacetimidate or trifluorophenylacetimidate rhamnose or arabinose donor under the promotion of a Lewis acid to yield a second glycosylated compound; and
(c) submitting the second glycosylated compound to deacetylation conditions.

48. The method of claim 47, wherein said Lewis acid is (i) trimethylsilyl trifluoromethanesulfonate (TMSOTf), (ii) tert-butyldimethylsilyl trifluoromethanesulfonate (TBSOTf), (iii) boron trifluoride diethyletherate (BF3-OEt₂), or (iv) any combination of (i) to (iii).

49. The method of claim 47, wherein the method comprises the steps of (1), and wherein said Lewis acid of (a) and/or (c) is (i) trimethylsilyl trifluoromethanesulfonate (TMSOTf), (ii) tert-butyldimethylsilyl trifluoromethanesulfonate (TBSOTf), (iii) boron trifluoride diethyletherate (BF₃-OEt₂), or (iv) any combination of (i) to (iii).

50. The method of claim 47, wherein the method comprises the steps of (1), and wherein said regioselective deacetylation conditions comprise (a) acetyl chloride (AcCl) in a solution of CH₂Cl₂/MeOH, (b) para-toluenesulfonic acid monohydrate (TsOH.H₂O) in a solution of CH₂Cl₂/MeOH at 40° C., or (c) hydrazine hydrate (NH₂NH₂.xH₂O) in tetrahydrofuran (THF).

51. The method of claim 47,
wherein the method comprises the steps of (1), and wherein said deacetylation conditions of (d) comprise (i) NaOMe and MeOH (Zemplén deacetylation conditions) or (ii) NaOH in MeOH/tetrahydrofuran/H₂O; or
wherein the method comprises the steps of (2) and wherein said deacetylation conditions of (c) comprise (i) NaOMe and MeOH (Zemplén deacetylation conditions) or (ii) NaOH in MeOH/tetrahydrofuran/H₂O.

52. The method of claim 51, wherein said NaOH is at about 0.5 N.

53. The method of claim 47, wherein the method comprises the steps of (1), and wherein said perbenzoylated or peracetylated trichloroacetimidate or trifluorophenylacetimidate glucose donor is 2,3,4,6-tetra-O-benzoyl-α-D-glucopyranosyl trichloroacetimidate.

54. The method of claim 47, wherein said perbenzoylated or peracetylated trichloroacetimidate or trifluorophenylacetimidate arabinose donor is 2,3,4-tri-O-benzoyl-β-L-arabinopyranosyl trichloroacetimidate.

55. The method of claim 47, wherein said perbenzoylated or peracetylated trichloroacetimidate or trifluorophenylacetimidate rhamnose donor is 2,3,4-tri-O-benzoyl-α-L-rhamnopyranosyl trichloroacetimidate.

56.-59. (canceled)

60. The method of claim 47, wherein the method comprises the steps of (2), and wherein said phase-transfer conditions comprises K₂CO₃, a quaternary ammonium salt, CH₂Cl₂/H₂O and reflux.

61. The method of claim 60, wherein said quaternary ammonium salt is Bu₄NI Bu₄NBr, Bu₄NCl, Aliquat™ 100, Aliquat™ 175, Aliquat™ 336 or Aliquat™ HTA-1.

62. The method of claim 47, wherein the method comprises the steps of (2), and wherein said perbenzoylated or peracetylated bromide glucose donor is 2,3,4,6-tetra-O-benzoyl-α-D-glucopyranosyl bromide.

63.-64. (canceled)

65. A method for preparing a compound of formula (I): 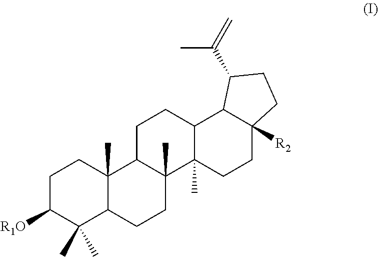
wherein
(i) R₁ is β-D-glucopyranose and R₂ is COO-β-D-glucopyranose or CH₂O-β-D-glucopyranose; or
(ii) R₁ is α-L-rhamnopyranose and R₂ is COO-β-L-rhamnopyranose or CH₂O-β-L-rhamnopyranose;
said method comprising
(a) glycosylating the C-3 and C-28 positions of betulin or betulinic acid with a perbenzoylated or peracetylated trichloroacetimidate or trifluorophenylacetimidate glucose or rhamnose donor under the promotion of a Lewis acid via a Schmidt's inverse procedure to yield a glycosylated compound; and
(b) submitting the glycosylated compound to deacetylation conditions.

66. The method of claim 65, wherein said Lewis acid is (i) trimethylsilyl trifluoromethanesulfonate (TMSOTf), (ii) tert-butyldimethylsilyl trifluoromethanesulfonate (TBSOTf), (iii) boron trifluoride diethyletherate (BF₃—OEt₂), or (iv) any combination of (i) to (iii).

67. The method of claim 65, wherein said deacetylation conditions comprise (i) NaOMe and MeOH (Zemplén deacetylation conditions) or (ii) NaOH in MeOH/tetrahydrofuran/H₂O.

68. The method of claim 65, wherein (a) glycosylates the C-3 and C-28 positions of betulin.

69. The method of claim 65, wherein (a) glycosylates the C-3 and C-28 positions of betulinic acid.

70. The method of claim 65, wherein said perbenzoylated or peracetylated trichloroacetimidate or trifluorophenylacetimidate glucose donor is 2,3,4,6-tetra-O-benzoyl-α-D-glucopyranosyl trichloroacetimidate.

71. The method of claim 65, wherein said perbenzoylated or peracetylated trichloroacetimidate or trifluorophenylacetimidate rhamnose donor is 2,3,4-tri-O-α-L-rhamnopyranosyl trichloroacetimidate.

72. The method of claim 65, wherein said Schmidt's inverse procedure comprises pre-mixing said betulin or betulinic acid with said Lewis acid before adding said perbenzoylated or peracetylated trichloroacetimidate or trifluorophenylacetimidate glucose or rhamnose donor.

73. The method of claim 72, wherein said addition of said perbenzoylated or peracetylated trichloroacetimidate or trifluorophenylacetimidate glucose or rhamnose donor is performed at a temperature of between about −78° C. to about 25° C.

74. The method of claim 73, wherein said addition of said perbenzoylated or peracetylated trichloroacetimidate or trifluorophenylacetimidate glucose or rhamnose donor is performed at a temperature of about −10° C.
